(19) United States
(12) Patent Application Publication
(10) Pub. No.: US 2026/0308126 A1
(43) Pub. Date: Oct. 8, 2026

(54) CRYSTALLINE FORMS OF 3-(5,7-DIFLUORO-4-OXO-1,4-DIHYDROQUINOLIN-2-YL)-4-(METHYLSULFONYL)BENZONITRILE

(71) Applicant: Flare Therapeutics Inc., Cambridge, MA (US)

(72) Inventors: Mark E. Scott, Cambridge, MA (US); Michael J. Geier, Cambridge, MA (US); Yaohui Huang, Shanghai (CN); Runyan Li, Shanghai (CN); Xiaoyang Wang, Shanghai (CN); Hong Deng, Shanghai (CN); Tingting Fan, Shanghai (CN)

(21) Appl. No.: 19/478,975

(22) PCT Filed: Apr. 26, 2024
(86) PCT No.: PCT/US2024/026635

§ 371 Date: Oct. 27, 2025

(30) Foreign Application Priority Data
Apr. 8, 2023 (WO) PCT/CN2023/091795

(51) Int. Cl. C07D 215/233; A61K 31/47; C07C 309/04

(52) U.S. Cl. CPC C07D 215/233; A61K 31/47; C07C 309/04; C07B 2200/13

(57) ABSTRACT

Provided are crystalline forms of 3-(5,7-difluoro-4-oxo-1,4-dihydroquinolin-2-yl)-4-(methylsulfonyl)benzonitrile, pharmaceutical compositions comprising the crystalline form, and their use in treating conditions associated with the suppression of PPARG cell signaling.

### RELATED APPLICATIONS

This application claims the benefit of priority to International Application No. PCT/CN2023/091795, filed Apr. 28, 2023, the entire contents of which are incorporated herein by reference.

### BACKGROUND

PPARgamma (PPARG) is a type II ligand-dependent nuclear hormone receptor (belonging to the PPAR nuclear receptor subfamily) that functions as an obligate heterodimer with retinoid X receptors (RXRs). PPARG is predominantly expressed in adipose tissue, colon, macrophages and the luminal layers of the urothelium. PPARG is known as a master regulator of adipogenesis, functioning to regulate adipocyte differentiation, fatty acid storage and glucose metabolism. PPARG has also been shown to play an important role in the metabolism and inflammation of macrophages, where it is induced by IL4 and controls glutamine metabolism. In the normal urothelium, PPARG is critical for its homeostasis and regeneration.

The role for PPARG in cancer was originally inferred from genomic studies that identified a PAX8-PPARG chromosomal rearrangement in follicular thyroid carcinomas. More recently, PPARG has been found to be over-expressed and genetically altered in the luminal subtype of urothelial cancer. This is consistent with reports that long-term use of PPARG agonists is associated with an increased incidence of urothelial cancer. Most urothelial cancers are urothelial carcinoma, which are classified as either non-muscle-invasive urothelial cancer (NMIUC, 70%), muscle-invasive urothelial cancer (MIUC, 25%) or metastatic urothelial cancer (MUC, 5%). MIUC is usually diagnosed de novo but may arise from the 10 to 20% of NMIUC cases that eventually progress. MIUC is a heterogeneous and aggressive disease, associated with a five-year survival rate of 60% for patients with localized disease and less than 10% for patients with distant metastases. Molecular understanding of NMIUC and MIUC has improved significantly, including the association between molecular subtypes and urothelial differentiation. Several molecular classes of MIUC have been proposed, whereby an activated PPARG signature features prominently in the luminal subtypes. First-line treatment is chemotherapy with several options in chemo-ineligible or second line, but treatment options are limited with poor overall survival rates.

Compound 1, 3-(5,7-difluoro-4-oxo-1,4-dihydroquinolin-2-yl)-4-(methylsulfonyl)benzonitrile, is a small molecule inverse agonist of PPARG showing great therapeutic potential for treating a variety of conditions associated with suppression of PPARG cell signaling, e.g., in treating proliferative disorders such as cancer. Compound 1 is exemplified in PCT/CN2022/129034, the contents of which is incorporated herein by reference. The development of alternative forms of Compound 1 represents an attractive area to further the treatment of diseases or disorders responsive to inhibition of EZH2.

### SUMMARY

Provided herein are crystalline forms of 3-(5,7-difluoro-4-oxo-1,4-dihydroquinolin-2-yl)-4-(methylsulfonyl)benzonitrile, having the following chemical structure:

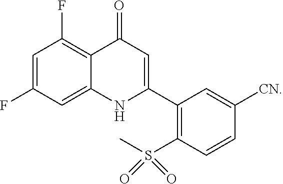 [SMILES: CS(=O)(=O)c1ccc(C#N)cc1-c1cc(=O)c2c(F)cc(F)cc2[nH]1]

Pharmaceutical compositions comprising the crystalline forms and methods for their preparation and use for treating conditions responsive to the modulation of PPARG are also included.

### BRIEF DESCRIPTION OF THE FIGURES

FIG. 1 depicts an X-ray powder diffraction pattern (XRPD) for crystalline Form B of 3-(5,7-difluoro-4-oxo-1,4-dihydroquinolin-2-yl)-4-(methylsulfonyl)benzonitrile.

FIG. 2 depicts a differential scanning calorimetry (DSC) thermogram for crystalline Form B of 3-(5,7-difluoro-4-oxo-1,4-dihydroquinolin-2-yl)-4-(methylsulfonyl)benzonitrile.

FIG. 3 depicts an X-ray powder diffraction pattern (XRPD) for crystalline Form C of 3-(5,7-difluoro-4-oxo-1,4-dihydroquinolin-2-yl)-4-(methylsulfonyl)benzonitrile.

FIG. 4 depicts a differential scanning calorimetry (DSC) thermogram for crystalline Form C of 3-(5,7-difluoro-4-oxo-1,4-dihydroquinolin-2-yl)-4-(methylsulfonyl)benzonitrile.

FIG. 5 depicts an X-ray powder diffraction pattern (XRPD) for crystalline Form D of 3-(5,7-difluoro-4-oxo-1,4-dihydroquinolin-2-yl)-4-(methylsulfonyl)benzonitrile.

FIG. 6 depicts a differential scanning calorimetry (DSC) thermogram for crystalline Form D of 3-(5,7-difluoro-4-oxo-1,4-dihydroquinolin-2-yl)-4-(methylsulfonyl)benzonitrile.

FIG. 7 depicts an X-ray powder diffraction pattern (XRPD) for crystalline Form E of 3-(5,7-difluoro-4-oxo-1,4-dihydroquinolin-2-yl)-4-(methylsulfonyl)benzonitrile.

FIG. 8 depicts a differential scanning calorimetry (DSC) thermogram for crystalline Form E of 3-(5,7-difluoro-4-oxo-1,4-dihydroquinolin-2-yl)-4-(methylsulfonyl)benzonitrile.

FIG. 9 depicts an X-ray powder diffraction pattern (XRPD) for crystalline Form F of 3-(5,7-difluoro-4-oxo-1,4-dihydroquinolin-2-yl)-4-(methylsulfonyl)benzonitrile.

FIG. 10 depicts a differential scanning calorimetry (DSC) thermogram for crystalline Form F of 3-(5,7-difluoro-4-oxo-1,4-dihydroquinolin-2-yl)-4-(methylsulfonyl)benzonitrile.

FIG. 11 depicts an X-ray powder diffraction pattern (XRPD) for crystalline Form G of 3-(5,7-difluoro-4-oxo-1,4-dihydroquinolin-2-yl)-4-(methylsulfonyl)benzonitrile.

FIG. 12 depicts a differential scanning calorimetry (DSC) thermogram for crystalline Form G of 3-(5,7-difluoro-4-oxo-1,4-dihydroquinolin-2-yl)-4-(methylsulfonyl)benzonitrile.

FIG. 13 depicts an X-ray powder diffraction pattern (XRPD) for crystalline Form G′ of 3-(5,7-difluoro-4-oxo-1,4-dihydroquinolin-2-yl)-4-(methylsulfonyl)benzonitrile.

FIG. 14 depicts a differential scanning calorimetry (DSC) thermogram for crystalline Form G′ of 3-(5,7-difluoro-4-oxo-1,4-dihydroquinolin-2-yl)-4-(methylsulfonyl)benzonitrile.

FIG. 15 depicts an X-ray powder diffraction pattern (XRPD) for crystalline Form H of 3-(5,7-difluoro-4-oxo-1,4-dihydroquinolin-2-yl)-4-(methylsulfonyl)benzonitrile.

FIG. 16 depicts an X-ray powder diffraction pattern (XRPD) for crystalline Form A of 3-(5,7-difluoro-4-oxo-1,4-dihydroquinolin-2-yl)-4-(methylsulfonyl)benzonitrile hydrochloride.

FIG. 17 depicts a differential scanning calorimetry (DSC) thermogram for crystalline Form A of 3-(5,7-difluoro-4-oxo-1,4-dihydroquinolin-2-yl)-4-(methylsulfonyl)benzonitrile hydrochloride.

FIG. 18 depicts an X-ray powder diffraction pattern (XRPD) for crystalline Form A of 3-(5,7-difluoro-4-oxo-1,4-dihydroquinolin-2-yl)-4-(methylsulfonyl)benzonitrile methanesulfonic acid salt.

FIG. 19 depicts a differential scanning calorimetry (DSC) thermogram for crystalline Form A of 3-(5,7-difluoro-4-oxo-1,4-dihydroquinolin-2-yl)-4-(methylsulfonyl)benzonitrile methanesulfonic acid salt.

### DETAILED DESCRIPTION

### 1. General Description of Compound Forms

Provided herein are crystalline Forms B, C, D, E, F, G, G′, and H of 3-(5,7-difluoro-4-oxo-1,4-dihydroquinolin-2-yl)-4-(methylsulfonyl)benzonitrile.

Also provided is crystalline Form A of 3-(5,7-difluoro-4-oxo-1,4-dihydroquinolin-2-yl)-4-(methylsulfonyl)benzonitrile hydrochloride.

Also provided is crystalline Form A of 3-(5,7-difluoro-4-oxo-1,4-dihydroquinolin-2-yl)-4-(methylsulfonyl)benzonitrile methanesulfonic acid salt.

### Definitions

As used herein, “crystalline” refers to a solid form of a compound wherein there exists long-range atomic order in the positions of the atoms. The crystalline nature of a solid can be confirmed, for example, by examination of the X-ray powder diffraction pattern. Unless otherwise specified, the crystalline forms (Form B, C, D, E, F, G, G′, H, crystalline Form A of Compound 1 hydrochloride, and crystalline Form A of Compound 1 methanesulfonic acid salt) are each single crystalline forms. A “single crystalline form” means that the recited compound, i.e., Compound 1, is present as a single crystal or a plurality of crystals in which each crystal has the same crystal form (e.g., B, C, D, E, F, G, G′, H, crystalline Form A of Compound 1 hydrochloride, or crystalline Form A of Compound 1 methanesulfonic acid salt). Percent by weight of a particular crystal form is determined by the weight of the particular crystal form divided by the sum weight of the particular crystal, plus the weight of the other crystal forms present plus the weight of amorphous form present multiplied by 100%.

The 2-theta values of the X-ray powder diffraction patterns for the crystalline form described herein may vary slightly from one instrument to another and also depending on variations in sample preparation and batch to batch variation due to factors such as temperature variation, sample displacement, and the presence or absence of an internal standard. Therefore, unless otherwise defined, the XRPD patterns/assignments recited herein are not to be construed as absolute and can vary ±0.2 degrees. It is well known in the art that this variability will account for the above factors without hindering the unequivocal identification of a crystal form. Unless otherwise specified, the 2-theta values provided herein were obtained using Cu Kα1 radiation.

Temperature values, e.g., for DSC peaks herein may vary slightly from one instrument to another and also depending on variations in sample preparation, batch to batch variation, heating rate of the method, and environmental factors. Therefore, unless otherwise defined, temperature values recited herein are not to be construed as absolute and can vary ±5 degrees or +2 degrees.

“Substantially the same XRPD pattern” or “an X-ray powder diffraction pattern substantially similar to” a defined figure means that for comparison purposes, at least 90% of the peaks shown are present. It is to be further understood that for comparison purposes some variability in peak intensities from those shown are allowed, such as +0.2 degrees.

The term “amorphous” refers to a solid that is present in a non-crystalline state or form. Amorphous solids are disordered arrangements of molecules and therefore possess no distinguishable crystal lattice or unit cell and consequently have no definable long range ordering. Solid state ordering of solids may be determined by standard techniques known in the art, e.g., by X-ray powder diffraction (XRPD) or differential scanning calorimetry (DSC).

The term “anhydrous” and “anhydrate” are used interchangeably and mean that the referenced crystalline form has substantially no water in the crystal lattice, e.g., less than 1% by weight as determined by Karl Fisher analysis

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a crystalline form described herein that will elicit a desired or beneficial biological or medical response of a subject e.g., a dosage of between 0.01-100 mg/kg body weight/day.

### Exemplary Forms

In a first embodiment, provided is crystalline Form B of a compound having the structural formula:

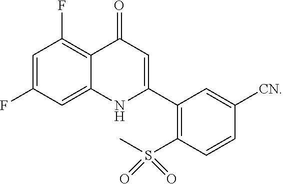 [SMILES: CS(=O)(=O)c1ccc(C#N)cc1-c1cc(=O)c2c(F)cc(F)cc2[nH]1]

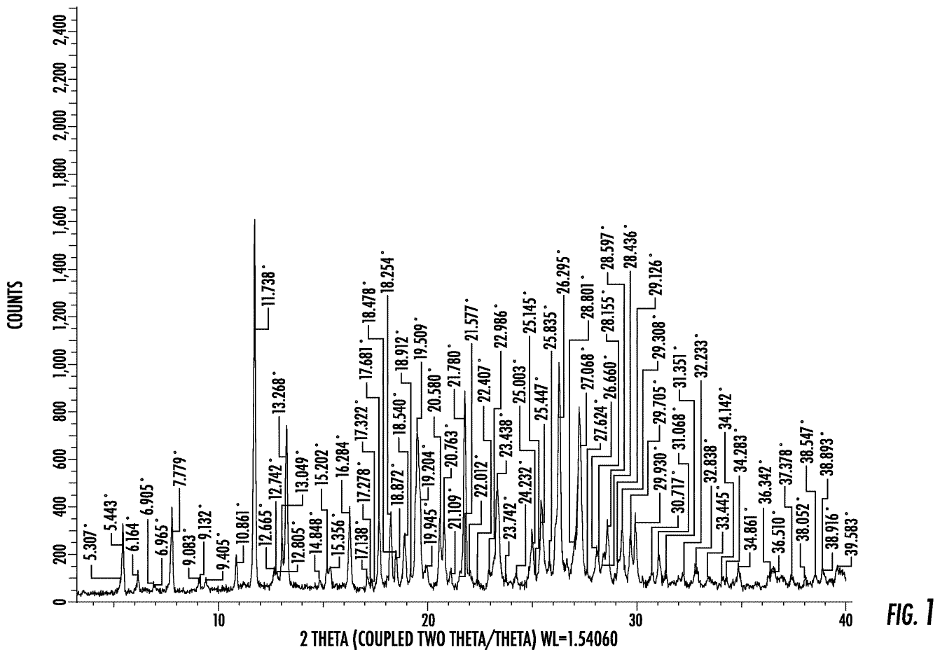

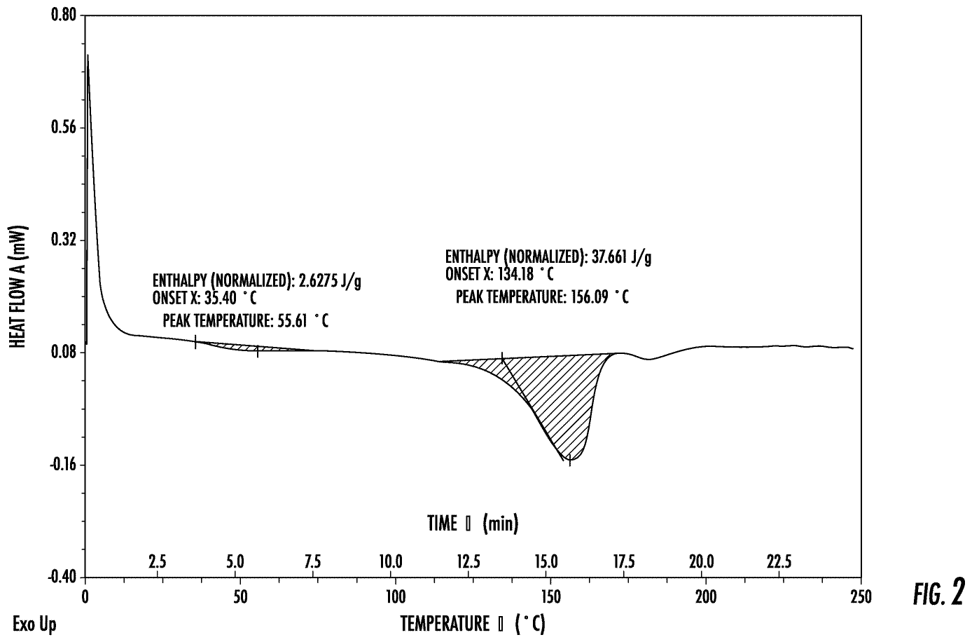

In a second embodiment, crystalline Form B is characterized by at least three, at least four, or at least five x-ray powder diffraction peaks at 2θ angles selected from 11.74°, 13.27°, 19.51°, 21.78°, 26.30°, and 27.26°. Alternatively, as part of a second embodiment, crystalline Form B is characterized by x-ray powder diffraction peaks at 2θ angles selected from 11.74°, 13.27°, 19.51°, 21.78°, 26.30°, and 27.26°. In another alternative, as part of a second embodiment, crystalline Form B is characterized by x-ray powder diffraction peaks at 2θ angles selected from 11.74°, 13.27°, 19.51°, 21.78°, 26.30°, and 27.26°, and at least one, at least two, at least three, at least four, at least five, at least six, at least seven, at least eight, or at least nine additional x-ray powder diffraction peaks at 2θ angles selected from 7.78°, 16.28°, 17.68°, 18.25°, 18.91°, 20.58°, 23.35°, 25.45°, 28.60°, and 29.93°. In yet another alternative, as part of a second embodiment, crystalline Form B is characterized by x-ray powder diffraction peaks at 2θ angles selected from 7.78°, 11.74°, 13.27°, 16.28°, 17.68°, 18.25°, 18.91°, 19.51°, 20.58°, 21.78°, 23.35°, 25.45°, 26.30°, 27.26°, 28.60°, and 29.93°. In yet another alternative, as part of a second embodiment, crystalline Form B is characterized by at least three, at least four, at least five, at least six, at least seven, at least eight, at least nine, at least ten, at least eleven, at least twelve, at least thirteen, or at least fourteen x-ray powder diffraction peaks at 2θ angles selected from those in Table 1. In yet another alternative, as part of a second embodiment, crystalline Form B is characterized by an XRPD substantially similar to FIG. 1. In yet another alternative, as part of a second embodiment, crystalline Form B is characterized by a DSC substantially similar to FIG. 2.

TABLE 1

| Position (°2θ) | Relative Intensity (%) |
|---|---|
| 5.307 | 4.00 |
| 5.443 | 16.70 |
| 6.164 | 5.50 |
| 6.905 | 3.90 |
| 6.965 | 2.70 |
| 7.779 | 23.40 |
| 9.083 | 4.90 |
| 9.132 | 6.10 |
| 9.405 | 3.30 |
| 10.861 | 8.70 |
| 11.738 | 100.00 |
| 12.665 | 7.30 |
| 12.742 | 3.80 |
| 12.805 | 5.50 |
| 13.049 | 18.60 |
| 13.268 | 43.90 |
| 14.848 | 4.50 |
| 15.202 | 5.50 |
| 15.356 | 8.00 |
| 16.284 | 23.10 |
| 17.138 | 3.10 |
| 17.278 | 3.40 |
| 17.322 | 2.30 |
| 17.681 | 26.00 |
| 18.254 | 28.50 |
| 18.478 | 13.10 |
| 18.54 | 10.60 |
| 18.872 | 18.90 |
| 18.912 | 20.20 |
| 19.204 | 3.70 |
| 19.509 | 60.10 |
| 19.945 | 5.40 |
| 20.58 | 27.50 |
| 20.763 | 19.90 |
| 21.109 | 4.90 |
| 21.577 | 3.90 |
| 21.78 | 58.80 |
| 22.012 | 4.30 |
| 22.408 | 1.90 |
| 22.986 | 3.10 |
| 23.346 | 38.10 |
| 23.742 | 1.70 |
| 24.232 | 3.50 |
| 25.003 | 15.00 |
| 25.145 | 2.80 |
| 25.447 | 22.50 |
| 25.835 | 5.60 |
| 26.295 | 62.30 |
| 26.66 | 6.70 |
| 27.068 | 11.60 |
| 27.262 | 48.70 |
| 27.624 | 2.70 |
| 28.081 | 12.50 |
| 28.155 | 11.70 |
| 28.436 | 6.00 |
| 28.597 | 20.50 |
| 29.126 | 6.50 |
| 29.308 | 15.00 |
| 29.705 | 14.00 |
| 29.93 | 22.30 |
| 30.717 | 3.20 |
| 31.068 | 8.60 |
| 31.351 | 1.20 |
| 32.233 | 3.30 |
| 32.838 | 4.00 |
| 33.445 | 3.00 |
| 34.142 | 2.80 |
| 34.283 | 2.40 |
| 34.861 | 6.60 |
| 36.342 | 1.90 |
| 36.51 | 5.40 |
| 37.378 | 2.80 |
| 38.052 | 3.60 |
| 38.547 | 1.80 |
| 38.893 | 5.90 |
| 38.916 | 2.80 |
| 39.583 | 5.40 |

In a third embodiment, crystalline Form B as described herein (e.g., in the first or second embodiment), is a solvate. Alternatively, as part of a third embodiment, crystalline Form B as described herein (e.g., in the first or second embodiment), is a methanol solvate.

In a fourth embodiment, provided is crystalline Form C of a compound having the structural formula:

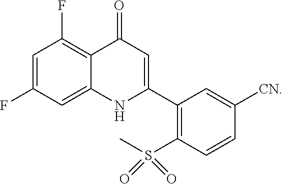 [SMILES: CS(=O)(=O)c1ccc(C#N)cc1-c1cc(=O)c2c(F)cc(F)cc2[nH]1]

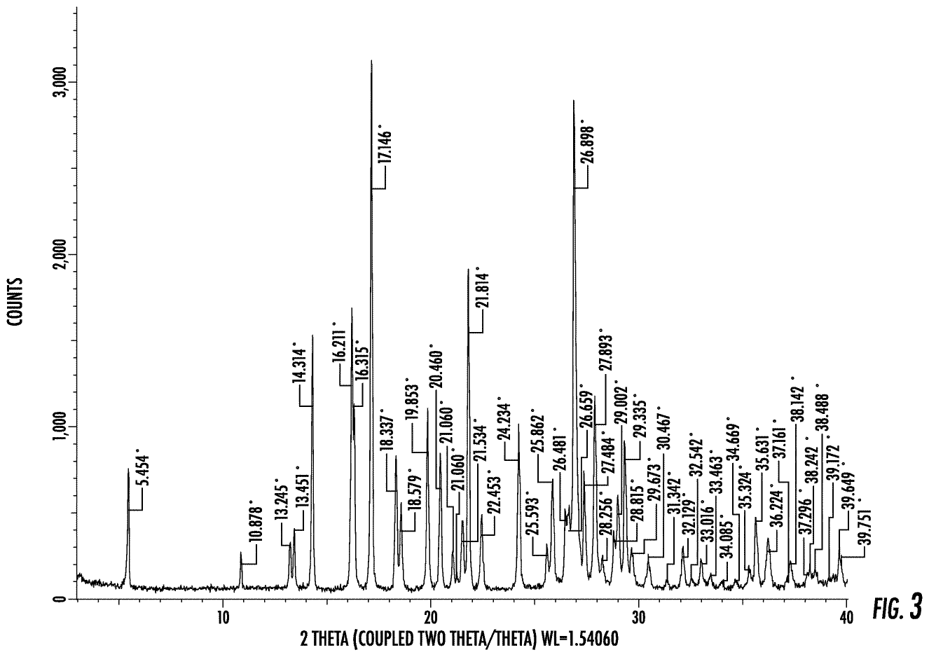

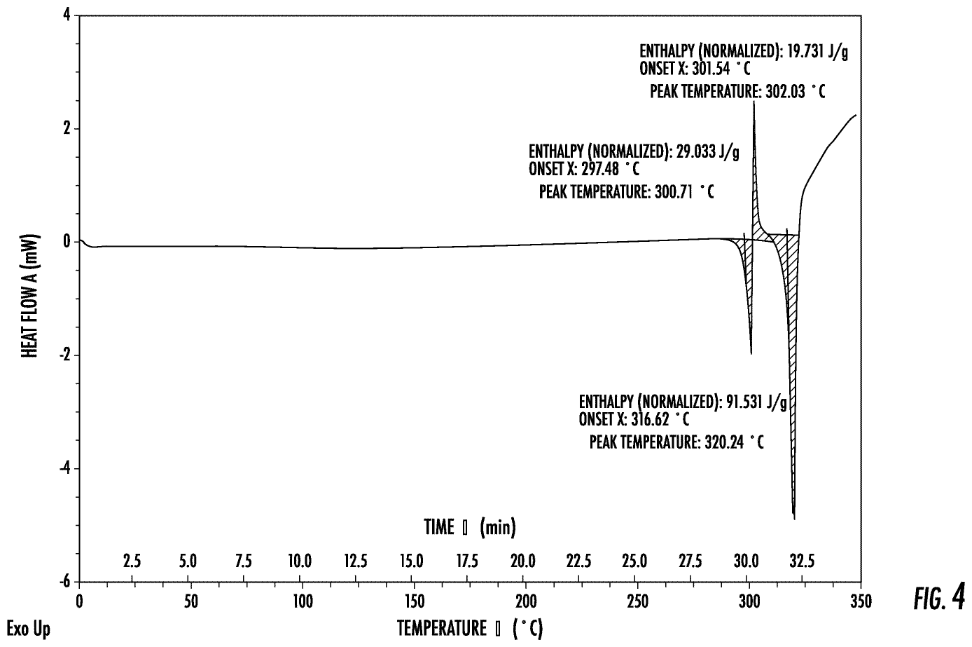

In a fifth embodiment, crystalline Form C is characterized by at least three, at least four, or at least five x-ray powder diffraction peaks at 2θ angles selected from 14.31°, 16.21°, 16.32°, 17.15°, 21.82°, and 26.90°. Alternatively, as part of a fifth embodiment, crystalline Form C is characterized by x-ray powder diffraction peaks at 2θ angles selected from 14.31°, 16.21°, 16.32°, 17.15°, 21.82°, and 26.90°. In another alternative, as part of a fifth embodiment, crystalline Form C is characterized by x-ray powder diffraction peaks at 2θ angles selected from 14.31°, 16.21°, 16.32°, 17.15°, 21.82°, and 26.90° and at least one, at least two, at least three, at least four, at least five, at least six, at and least seven, additional x-ray powder diffraction peaks at 2θ angles selected from 18.34°, 19.85°, 20.46°, 24.23°, 25.86°, 27.38°, 27.89°, and 29.34°. In yet another alternative, as part of a fifth embodiment, crystalline Form C is characterized by x-ray powder diffraction peaks at 2θ angles selected from 14.31°, 16.21°, 16.32°, 17.15°, 18.34°, 19.85°, 20.46°, 21.82°, 24.23°, 25.86°, 26.90°, 27.38°, 27.89°, and 29.34°. In yet another alternative, as part of a fifth embodiment, crystalline Form C is characterized by at least three, at least four, at least five, at least six, at least seven, at least eight, at least nine, at least ten, at least eleven, at least twelve, at least thirteen, or at least fourteen x-ray powder diffraction peaks at 2θ angles selected from those in Table 2. In yet another alternative, as part of a fifth embodiment, crystalline Form C is characterized by an XRPD substantially similar to FIG. 3. In yet another alternative, as part of a fourth embodiment, crystalline Form C is characterized by a DSC substantially similar to FIG. 4.

In a sixth embodiment, crystalline Form C, as described herein (e.g., in the fourth or fifth embodiment) is anhydrous.

TABLE 2

| Position (°2θ) | Relative Intensity (%) |
|---|---|
| 5.454 | 20.10 |
| 10.878 | 6.40 |
| 13.245 | 11.40 |
| 13.451 | 14.80 |
| 14.314 | 46.00 |
| 16.211 | 50.50 |
| 16.315 | 46.50 |
| 17.146 | 100.00 |
| 18.337 | 24.30 |
| 18.579 | 15.50 |
| 19.853 | 34.50 |
| 20.46 | 25.00 |
| 21.06 | 9.00 |
| 21.264 | 3.70 |
| 21.534 | 11.90 |
| 21.815 | 62.80 |
| 22.453 | 13.80 |
| 24.234 | 32.60 |
| 25.593 | 8.00 |
| 25.862 | 26.20 |
| 26.481 | 14.70 |
| 26.659 | 12.80 |
| 26.898 | 99.20 |
| 27.384 | 23.50 |
| 27.893 | 37.80 |
| 28.256 | 5.70 |
| 28.815 | 10.50 |
| 29.002 | 17.90 |
| 29.335 | 30.80 |
| 29.673 | 7.30 |
| 30.467 | 7.10 |
| 31.342 | 1.40 |
| 32.129 | 7.80 |
| 32.542 | 1.90 |
| 33.016 | 5.10 |
| 33.463 | 2.30 |
| 34.085 | 1.90 |
| 34.669 | 1.30 |
| 35.324 | 4.00 |
| 35.631 | 15.90 |
| 36.224 | 9.20 |
| 37.161 | 0.90 |
| 37.296 | 5.70 |
| 38.142 | 2.30 |
| 38.242 | 3.80 |
| 38.488 | 3.50 |
| 39.172 | 1.10 |
| 39.649 | 8.60 |
| 39.751 | 7.00 |

In a seventh embodiment, provided is crystalline Form D of a compound having the structural formula:

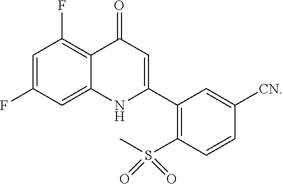 [SMILES: CS(=O)(=O)c1ccc(C#N)cc1-c1cc(=O)c2c(F)cc(F)cc2[nH]1]

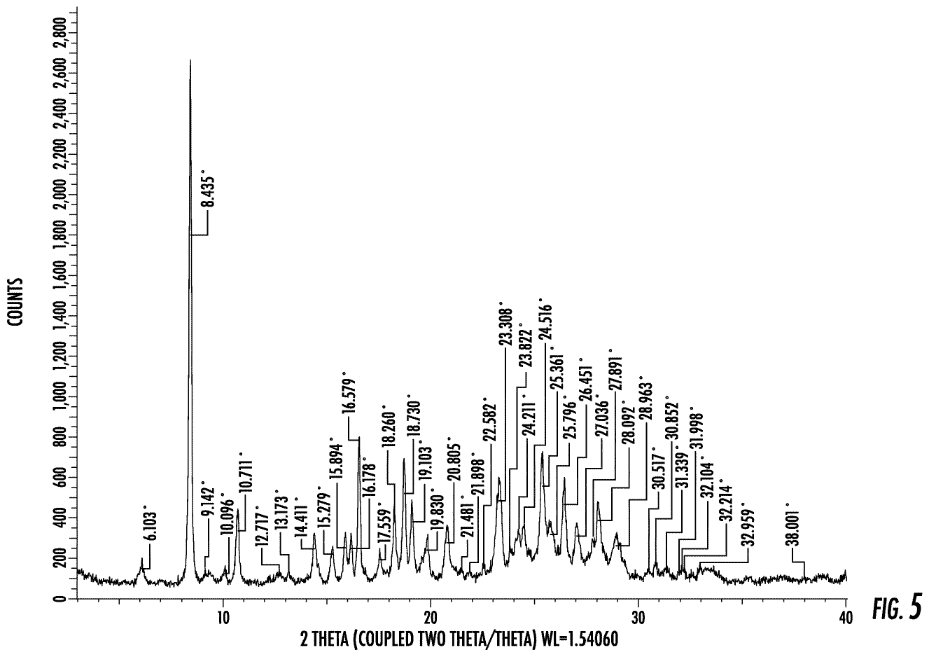

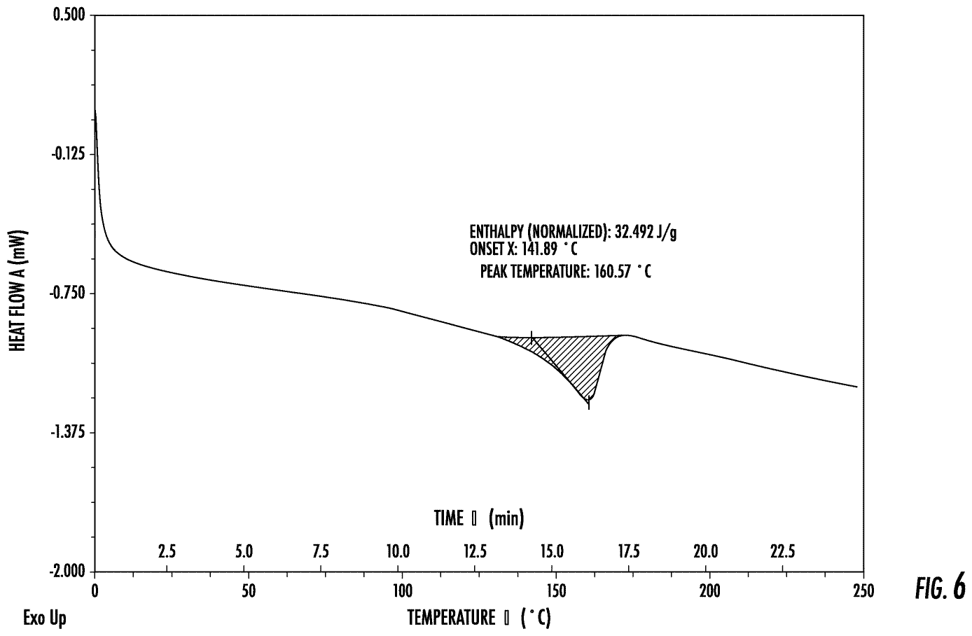

In an eighth embodiment, crystalline Form D is characterized by at least three, at least four, at least five, or at least six x-ray powder diffraction peaks at 2θ angles selected from 8.44°, 16.58°, 18.73°, 19.10°, 23.31°, 25.36°, and 26.45°. Alternatively, as part of an eighth embodiment, crystalline Form D is characterized by x-ray powder diffraction peaks at 2θ angles selected from 8.44°, 16.58°, 18.73°, 19.10°, 23.31°, 25.36°, and 26.45°. In another alternative, as part of an eighth embodiment, crystalline Form D is characterized by at least three, at least four, at least five, at least six, at least seven, at least eight, at least nine, at least ten, at least eleven, at least twelve, at least thirteen, or at least fourteen x-ray powder diffraction peaks at 2θ angles selected from those in Table 3. In yet another alternative, as part of an eighth embodiment, crystalline Form D is characterized by an XRPD substantially similar to FIG. 5. In yet another alternative, as part of an eighth embodiment, crystalline Form D is characterized by a DSC substantially similar to FIG. 6.

In a ninth embodiment, crystalline Form D, as described herein (e.g., in the seventh or eighth embodiment) is a solvate. In another alternative, as part of a ninth embodiment, crystalline Form D, as described herein (e.g., in the seventh or eighth embodiment) is an MBTE solvate.

TABLE 3

| Position (°2θ) | Relative Intensity (%) |
|---|---|
| 6.103 | 4.50 |
| 8.435 | 100.00 |
| 9.142 | 2.40 |
| 10.096 | 2.50 |
| 10.711 | 14.70 |
| 12.717 | 3.00 |
| 13.173 | 2.40 |
| 14.411 | 9.60 |
| 15.279 | 6.60 |
| 15.894 | 8.80 |
| 16.178 | 9.20 |
| 16.579 | 41.00 |
| 17.559 | 5.90 |
| 18.26 | 14.80 |
| 18.73 | 23.50 |
| 19.103 | 15.60 |
| 19.83 | 8.20 |
| 20.805 | 9.70 |
| 21.481 | 1.60 |
| 21.898 | 1.60 |
| 22.562 | 2.00 |
| 23.308 | 19.00 |
| 23.822 | 6.00 |
| 24.211 | 10.70 |
| 24.516 | 12.20 |
| 25.361 | 22.60 |
| 25.796 | 7.40 |
| 26.451 | 16.80 |
| 27.036 | 8.20 |
| 27.891 | 8.30 |
| 28.092 | 12.60 |
| 28.963 | 7.00 |
| 30.517 | 2.00 |
| 30.852 | 3.50 |
| 31.339 | 1.80 |
| 31.998 | 1.20 |
| 32.104 | 3.40 |
| 32.214 | 1.90 |
| 32.959 | 3.40 |
| 38.001 | 1.50 |

In a tenth embodiment, provided is crystalline Form E of a compound having the structural formula:

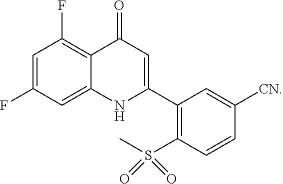 [SMILES: CS(=O)(=O)c1ccc(C#N)cc1-c1cc(=O)c2c(F)cc(F)cc2[nH]1]

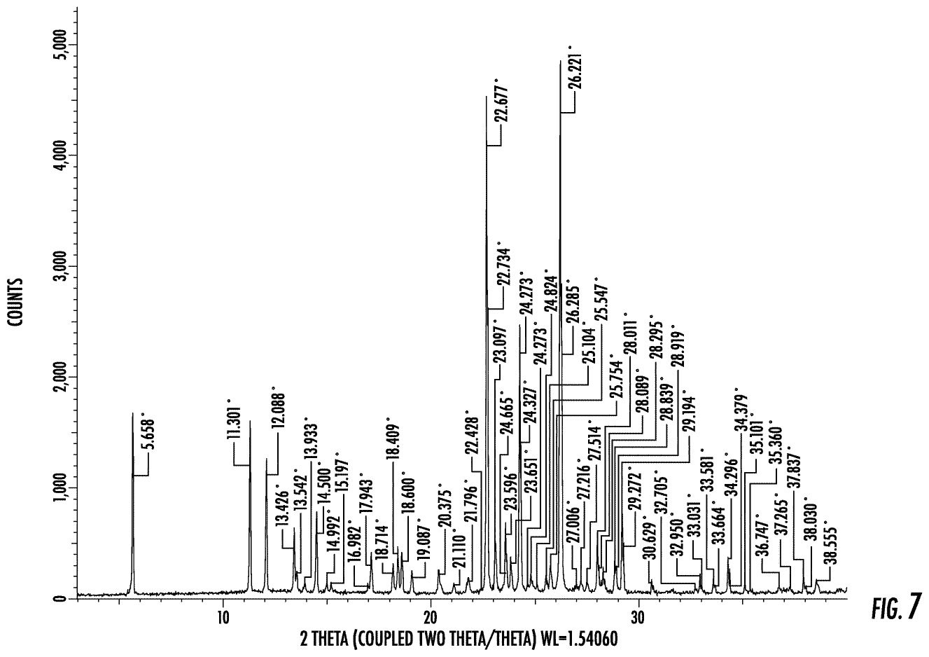

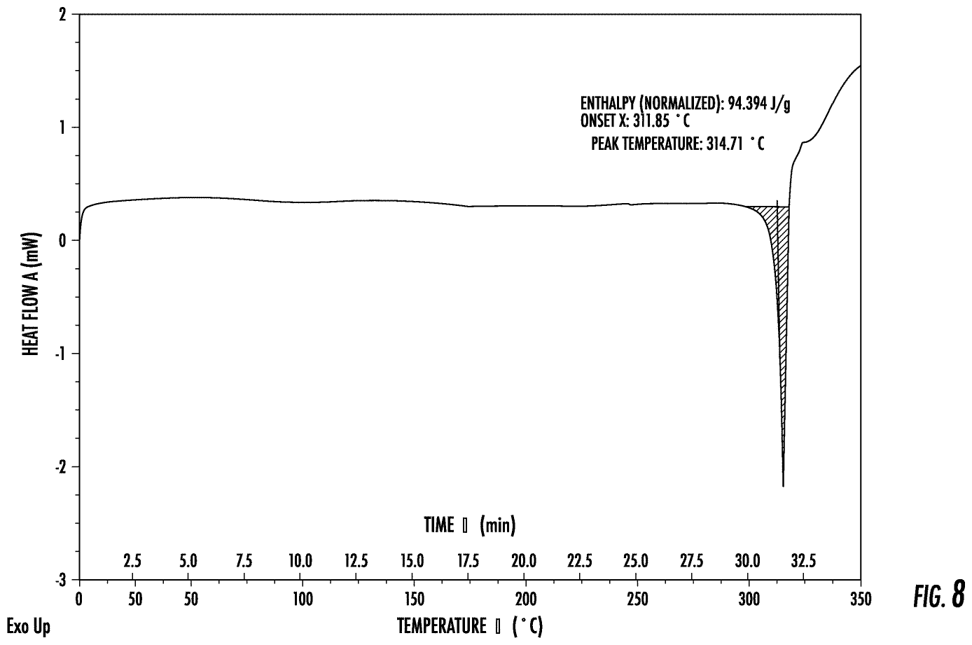

In an eleventh embodiment, crystalline Form E is characterized by at least three or at least four x-ray powder diffraction peaks at 2θ angles selected from 22.68°, 22.73°, 24.27°, 26.22°, and 26.29°. Alternatively, as part of an eleventh embodiment, crystalline Form E is characterized by x-ray powder diffraction peaks at 2θ angles selected from 22.68°, 22.73°, 24.27°, 26.22°, and 26.29°. In yet another alternative, as part of an eleventh embodiment, crystalline Form E is characterized by x-ray powder diffraction peaks at 2θ angles selected from 22.68°, 22.73°, 24.27°, 26.22°, and 26.29°, and at least one, at least two, at least, or at least four additional x-ray powder diffraction peaks at 2θ angles selected from 5.66°, 11.30°, 12.09°, 24.33°, and 29.19. In yet another alternative, as part of an eleventh embodiment, crystalline Form E is characterized by x-ray powder diffraction peaks at 2θ angles selected from 5.66°, 11.30°, 12.09°, 22.68°, 22.73°, 24.27°, 24.33°, 26.22°, 26.29°, and 29.19. In yet another alternative, as part of an eleventh embodiment, crystalline Form E is characterized by at least three, at least four, at least five, at least six, at least seven, at least eight, at least nine, at least ten, at least eleven, at least twelve, at least thirteen, or at least fourteen x-ray powder diffraction peaks at 2θ angles selected from those in Table 4. In yet another alternative, as part of an eleventh embodiment, crystalline Form E is characterized by an XRPD substantially similar to FIG. 7. In yet another alternative, as part of an eleventh embodiment, crystalline Form E is characterized by a DSC substantially similar to FIG. 8.

In a twelfth embodiment, crystalline Form E as described herein (e.g., in the tenth or eleventh embodiment), is anhydrous.

TABLE 4

| Position (°2θ) | Relative Intensity (%) |
|---|---|
| 5.658 | 24.90 |
| 11.301 | 26.30 |
| 12.088 | 19.80 |
| 13.426 | 9.50 |
| 13.542 | 4.10 |
| 13.933 | 1.50 |
| 14.5 | 12.60 |
| 14.992 | 1.80 |
| 15.197 | 1.60 |
| 16.982 | 1.30 |
| 17.132 | 6.40 |
| 18.179 | 4.70 |
| 18.409 | 7.70 |
| 18.6 | 6.90 |
| 19.087 | 3.40 |
| 20.375 | 4.00 |
| 21.11 | 1.60 |
| 21.796 | 2.30 |
| 22.428 | 0.40 |
| 22.677 | 91.70 |
| 22.734 | 58.10 |
| 23.097 | 9.20 |
| 23.596 | 12.20 |
| 23.651 | 3.10 |
| 23.844 | 5.90 |
| 24.273 | 48.20 |
| 24.327 | 30.00 |
| 24.665 | 1.00 |
| 24.824 | 2.80 |
| 25.104 | 1.40 |
| 25.547 | 2.60 |
| 25.754 | 4.20 |
| 26.221 | 100.00 |
| 26.285 | 48.50 |
| 27.006 | 1.40 |
| 27.216 | 2.00 |
| 27.514 | 1.70 |
| 28.011 | 8.70 |
| 28.089 | 5.30 |
| 28.295 | 4.10 |
| 28.839 | 6.70 |
| 28.919 | 5.30 |
| 29.194 | 17.20 |
| 29.272 | 10.10 |
| 30.629 | 2.40 |
| 32.705 | 1.10 |
| 32.95 | 3.70 |
| 33.031 | 2.40 |
| 33.581 | 3.10 |
| 33.664 | 1.60 |
| 34.296 | 7.10 |
| 34.379 | 1.60 |
| 35.101 | 1.60 |
| 35.36 | 0.90 |
| 36.747 | 1.10 |
| 37.265 | 0.90 |
| 37.937 | 3.00 |
| 38.03 | 1.60 |
| 38.555 | 2.50 |

In a thirteenth embodiment, provided is crystalline Form F of a compound having the structural formula:

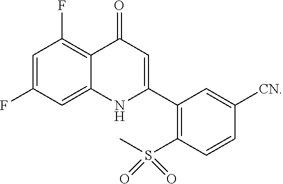 [SMILES: CS(=O)(=O)c1ccc(C#N)cc1-c1cc(=O)c2c(F)cc(F)cc2[nH]1]

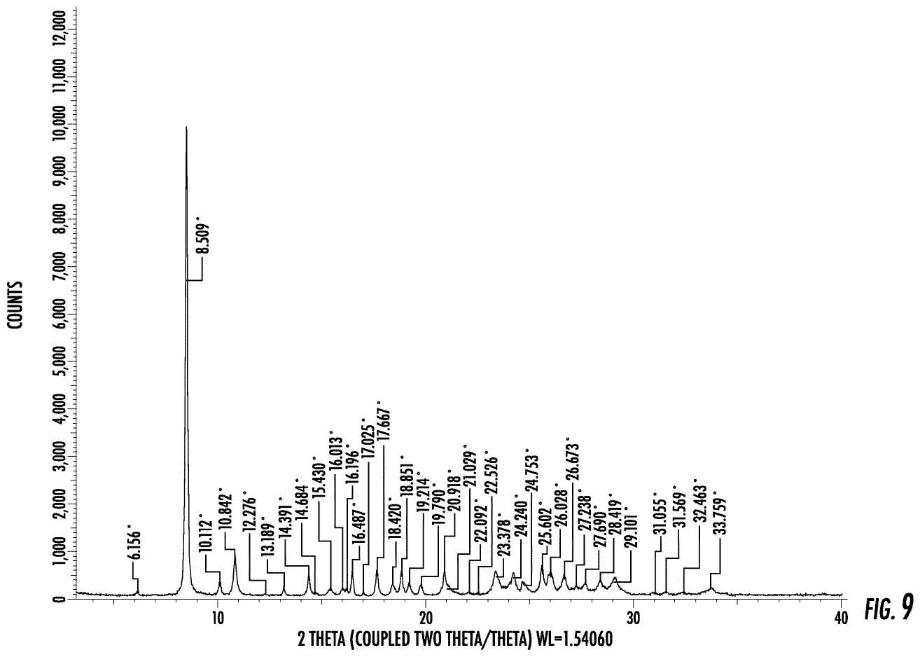

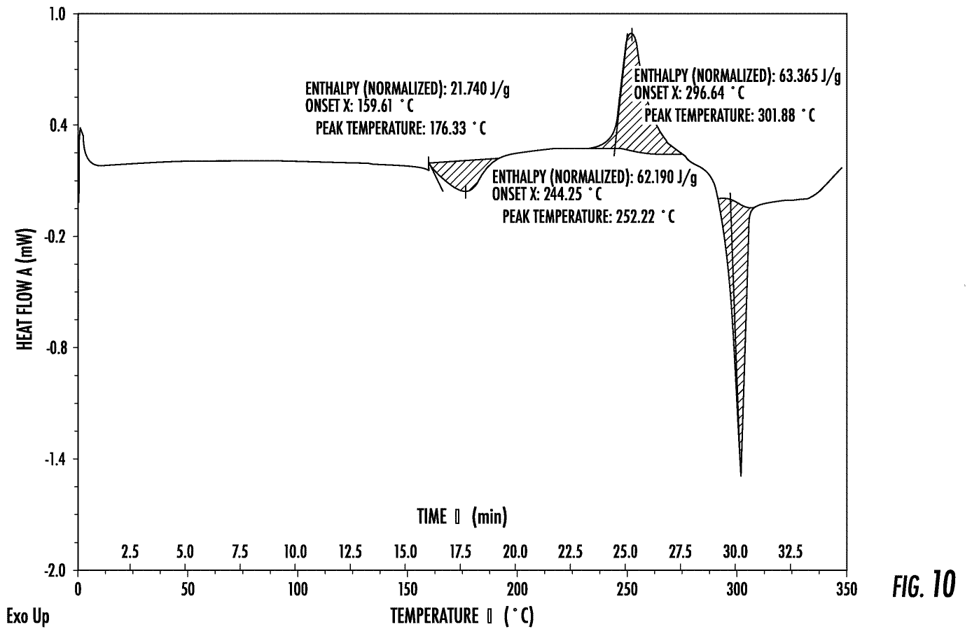

In a fourteenth embodiment, crystalline Form F is characterized by at least three, at least four, at least five, or at least six x-ray powder diffraction peaks at 2θ angles selected from 8.51°, 10.84°, 16.49°, 17.67°, 18.85°, 20.92°, and 25.60°. Alternatively, as part of a fourteenth embodiment, crystalline Form F is characterized by x-ray powder diffraction peaks at 2θ angles selected from 8.51°, 10.84°, 16.49°, 17.67°, 18.85°, 20.92°, and 25.60°. In another alternative, as part of a fourteenth embodiment, crystalline Form F is characterized by at least three, at least four, at least five, at least six, at least seven, at least eight, at least nine, at least ten, at least eleven, at least twelve, at least thirteen, or at least fourteen x-ray powder diffraction peaks at 2θ angles selected from those in Table 5. In yet another alternative, as part of a fourteenth embodiment, crystalline Form F is characterized by an XRPD substantially similar to FIG. 9. In yet another alternative, as part of a fourteenth embodiment, crystalline Form F is characterized by a DSC substantially similar to FIG. 10.

In a fifteenth embodiment, crystalline Form F as described herein e.g., (in the thirteenth or fourteenth embodiment) is a solvate. Alternatively, as part of a fifteenth embodiment, crystalline Form F as described herein e.g., (in the thirteenth or fourteenth embodiment) is a MeTHF solvate.

TABLE 5

| Position (°2θ) | Relative Intensity (%) |
|---|---|
| 6.156 | 1.00 |
| 8.509 | 100.00 |
| 10.112 | 2.40 |
| 10.842 | 8.70 |
| 12.276 | 0.30 |
| 13.189 | 2.20 |
| 14.391 | 4.40 |
| 14.684 | 0.70 |
| 15.43 | 1.50 |
| 16.013 | 1.70 |
| 16.196 | 1.30 |
| 16.487 | 5.80 |
| 17.025 | 0.60 |
| 17.667 | 5.60 |
| 18.42 | 2.50 |
| 18.851 | 5.20 |
| 19.214 | 2.60 |
| 19.79 | 2.30 |
| 20.918 | 7.70 |
| 21.029 | 1.90 |
| 22.092 | 0.50 |
| 22.526 | 0.40 |
| 23.378 | 4.90 |
| 24.24 | 4.10 |
| 24.753 | 1.80 |
| 25.602 | 6.10 |
| 26.028 | 4.60 |
| 26.673 | 3.90 |
| 27.238 | 1.00 |
| 27.69 | 1.80 |
| 28.419 | 2.40 |
| 29.101 | 3.40 |
| 31.055 | 0.40 |
| 31.569 | 0.70 |
| 32.463 | 0.30 |
| 33.759 | 1.50 |

In a sixteenth embodiment, provided is crystalline Form G of a compound having the structural formula:

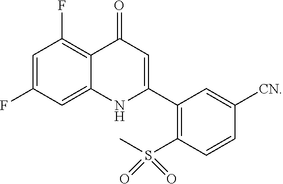 [SMILES: CS(=O)(=O)c1ccc(C#N)cc1-c1cc(=O)c2c(F)cc(F)cc2[nH]1]

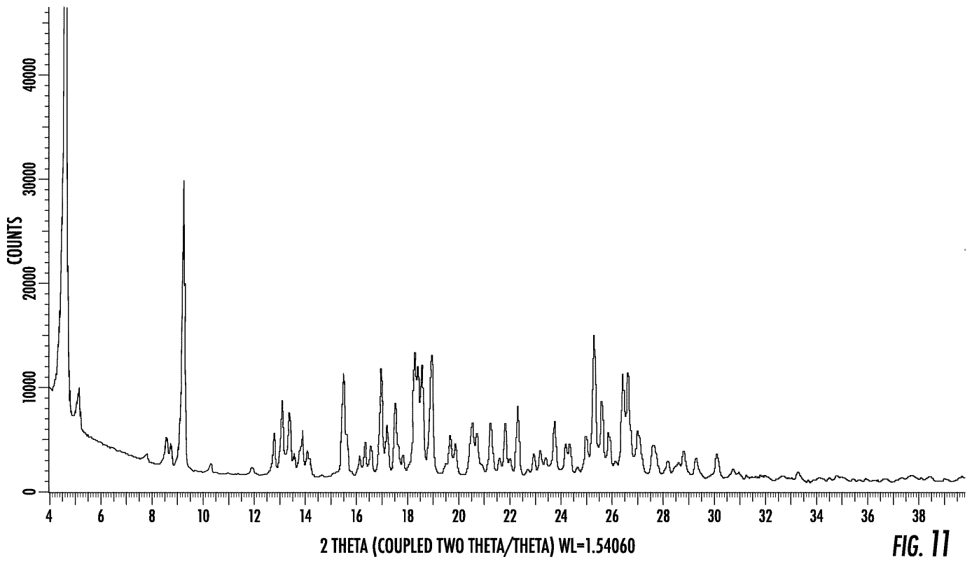

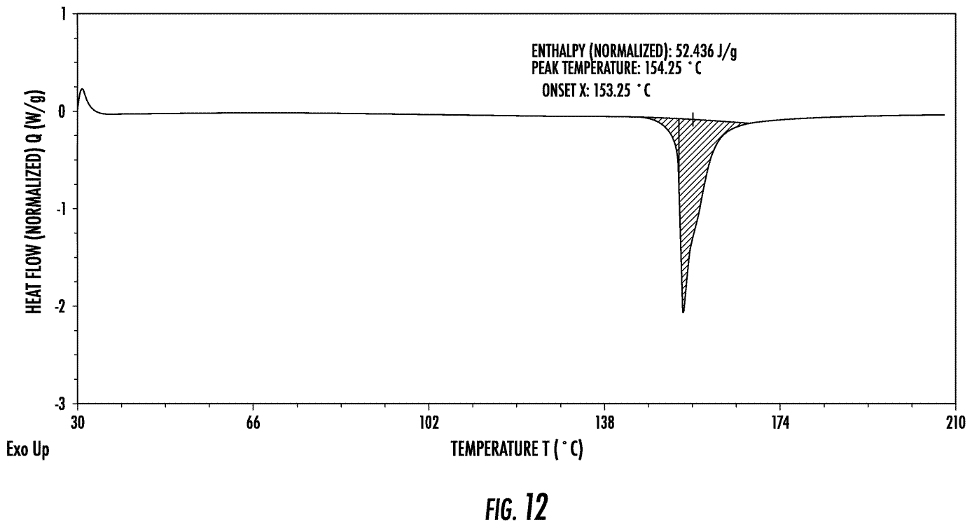

In a seventeenth embodiment, crystalline Form G is characterized by at least three, at least four, at least five, or at least six x-ray powder diffraction peaks at 2θ angles selected from 4.64°, 9.27°, 16.98°, 18.30°, 18.42°, 18.97°, and 25.29°. Alternatively, as part of a seventeenth embodiment, crystalline Form G is characterized by x-ray powder diffraction peaks at 2θ angles selected from 4.64°, 9.27°, 16.98°, 18.30°, 18.42°, 18.97°, and 25.29°. In another alternative, as part of a seventeenth embodiment, crystalline Form G is characterized by at least three, at least four, at least five, at least six, at least seven, at least eight, at least nine, at least ten, at least eleven, at least twelve, at least thirteen, or at least fourteen x-ray powder diffraction peaks at 2θ angles selected from those in Table 6. In yet another alternative, as part of a seventeenth embodiment, crystalline Form G is characterized by an XRPD substantially similar to FIG. 11. In yet another alternative, as part of a seventeenth embodiment, crystalline Form G is characterized by a DSC substantially similar to FIG. 12.

In an eighteenth embodiment, crystalline Form G as described herein e.g., (in the sixteenth or seventeenth embodiment) is a solvate. Alternatively, as part of an eighteenth embodiment, crystalline Form G as described herein e.g., (in the sixteenth or seventeenth embodiment) is a DMSO solvate.

TABLE 6

| Position (°2θ) | Relative Intensity (%) |
|---|---|
| 4.64 | 100 |
| 5.17 | 5 |
| 8.60 | 5 |
| 8.76 | 4 |
| 9.27 | 51 |
| 10.33 | 2 |
| 12.81 | 8 |
| 13.11 | 14 |
| 13.41 | 11 |
| 13.58 | 4 |
| 13.82 | 4 |
| 13.90 | 8 |
| 14.10 | 4 |
| 14.19 | 3 |
| 15.51 | 19 |
| 15.63 | 8 |
| 16.15 | 4 |
| 16.36 | 6 |
| 16.59 | 5 |
| 16.98 | 20 |
| 17.23 | 9 |
| 17.54 | 13 |
| 17.85 | 3 |
| 18.30 | 22 |
| 18.30 | 22 |
| 18.42 | 20 |
| 18.58 | 20 |
| 18.97 | 22 |
| 19.52 | 2 |
| 19.69 | 8 |
| 19.89 | 6 |
| 20.56 | 10 |
| 20.73 | 8 |
| 20.91 | 2 |
| 21.26 | 10 |
| 21.62 | 3 |
| 21.84 | 10 |
| 22.03 | 3 |
| 22.34 | 14 |
| 22.95 | 4 |
| 23.20 | 4 |
| 23.43 | 3 |
| 23.77 | 10 |
| 24.21 | 6 |
| 24.36 | 6 |
| 25.00 | 7 |
| 25.29 | 29 |
| 25.62 | 14 |
| 25.88 | 8 |
| 26.14 | 2 |
| 26.44 | 20 |
| 26.63 | 20 |
| 27.01 | 9 |
| 27.65 | 5 |
| 28.19 | 3 |
| 28.61 | 3 |
| 28.83 | 5 |
| 29.31 | 4 |
| 30.11 | 5 |
| 30.76 | 2 |
| 33.29 | 2 |

In a nineteenth embodiment, provided is crystalline Form G′ of a compound having the structural formula:

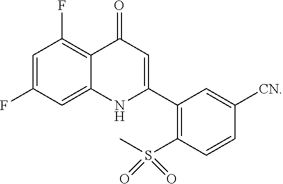 [SMILES: CS(=O)(=O)c1ccc(C#N)cc1-c1cc(=O)c2c(F)cc(F)cc2[nH]1]

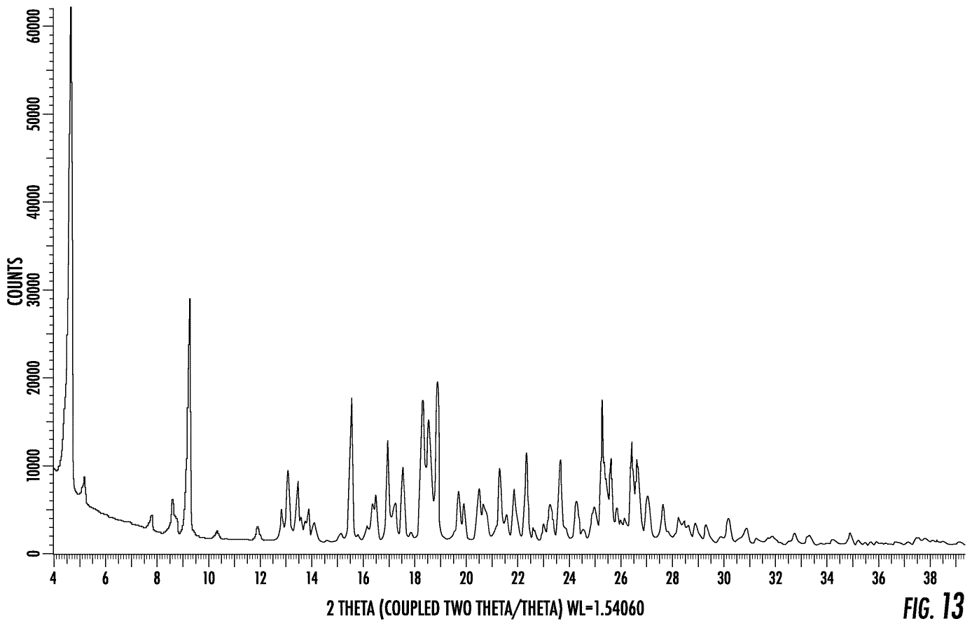

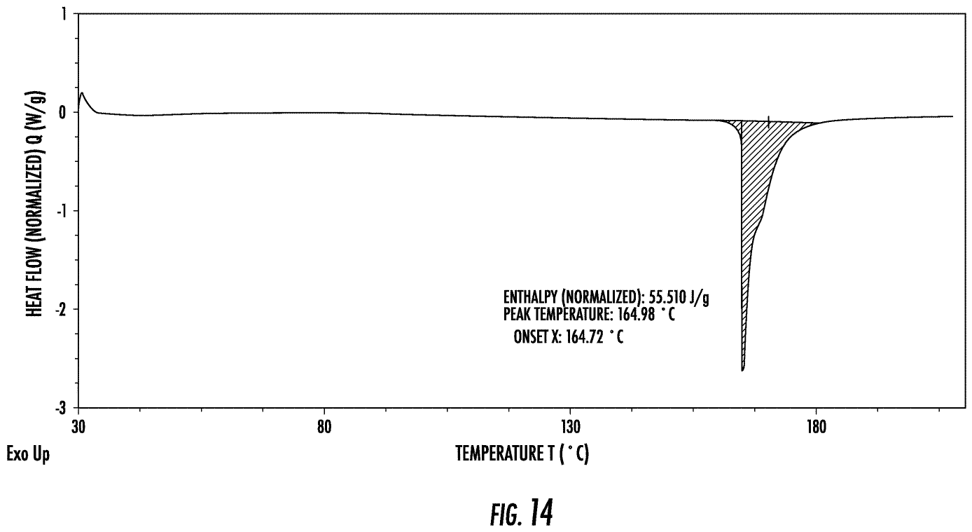

In a twentieth embodiment, crystalline Form G′ is characterized by at least three, at least four, at least five, or at least six x-ray powder diffraction peaks at 2θ angles selected from 4.65°, 9.26°, 15.55°, 18.31°, 18.87°, 25.27°, and 26.41°. Alternatively, as part of a twentieth embodiment, crystalline Form G′ is characterized by x-ray powder diffraction peaks at 2θ angles selected from 4.65°, 9.26°, 15.55°, 18.31°, 18.87°, 25.27°, and 26.41°. In another alternative, as part of a twentieth embodiment, crystalline Form G′ is characterized by at least three, at least four, at least five, at least six, at least seven, at least eight, at least nine, at least ten, at least eleven, at least twelve, at least thirteen, or at least fourteen x-ray powder diffraction peaks at 2θ angles selected from those in Table 7. In yet another alternative, as part of a twentieth embodiment, crystalline Form G′ is characterized by an XRPD substantially similar to FIG. 13. In yet another alternative, as part of a twentieth embodiment, crystalline Form F is characterized by a DSC substantially similar to FIG. 14.

In a twenty-first embodiment, crystalline Form G′ as described herein e.g., (in the nineteenth or twentieth embodiment) is a solvate. Alternatively, as part of a twenty-first embodiment, crystalline Form G′ as described herein e.g., (in the nineteenth or twentieth embodiment) is a DMSO solvate.

TABLE 7

| Position (°2θ) | Relative Intensity (%) |
|---|---|
| 4.65 | 100 |
| 5.18 | 5 |
| 7.79 | 3 |
| 8.62 | 8 |
| 8.76 | 5 |
| 9.26 | 56 |
| 11.90 | 3 |
| 12.84 | 8 |
| 13.08 | 17 |
| 13.45 | 15 |
| 13.60 | 6 |
| 13.76 | 5 |
| 13.89 | 7 |
| 14.11 | 4 |
| 15.14 | 2 |
| 15.18 | 2 |
| 15.55 | 35 |
| 15.78 | 2 |
| 16.16 | 4 |
| 16.37 | 9 |
| 16.49 | 12 |
| 16.97 | 25 |
| 17.19 | 8 |
| 17.26 | 9 |
| 17.54 | 18 |
| 17.86 | 2 |
| 18.31 | 35 |
| 18.54 | 29 |
| 18.61 | 17 |
| 18.87 | 40 |
| 19.71 | 13 |
| 19.71 | 13 |
| 19.91 | 9 |
| 20.50 | 13 |
| 20.67 | 9 |
| 20.73 | 6 |
| 21.13 | 3 |
| 21.13 | 3 |
| 21.30 | 19 |
| 21.57 | 6 |
| 21.85 | 14 |
| 22.34 | 23 |
| 22.62 | 3 |
| 23.00 | 5 |
| 23.25 | 8 |
| 23.65 | 23 |
| 23.86 | 2 |
| 24.28 | 9 |
| 24.55 | 2 |
| 24.88 | 7 |
| 24.98 | 8 |
| 25.27 | 40 |
| 25.41 | 15 |
| 25.61 | 23 |
| 25.85 | 8 |
| 26.00 | 5 |
| 26.16 | 6 |
| 26.41 | 27 |
| 26.61 | 22 |
| 27.02 | 11 |
| 27.61 | 10 |
| 27.81 | 2 |
| 28.13 | 2 |
| 28.25 | 6 |
| 28.46 | 5 |
| 28.63 | 4 |
| 28.88 | 6 |
| 29.31 | 5 |
| 29.89 | 2 |
| 30.18 | 6 |
| 30.88 | 4 |
| 31.72 | 2 |
| 31.90 | 2 |
| 32.73 | 3 |
| 33.23 | 2 |
| 33.33 | 2 |
| 34.90 | 4 |
| 37.47 | 2 |

In a twenty-second embodiment, provided is crystalline Form H of a compound having the structural formula:

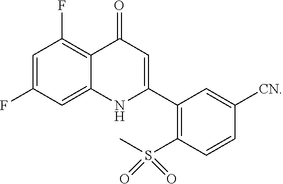 [SMILES: CS(=O)(=O)c1ccc(C#N)cc1-c1cc(=O)c2c(F)cc(F)cc2[nH]1]

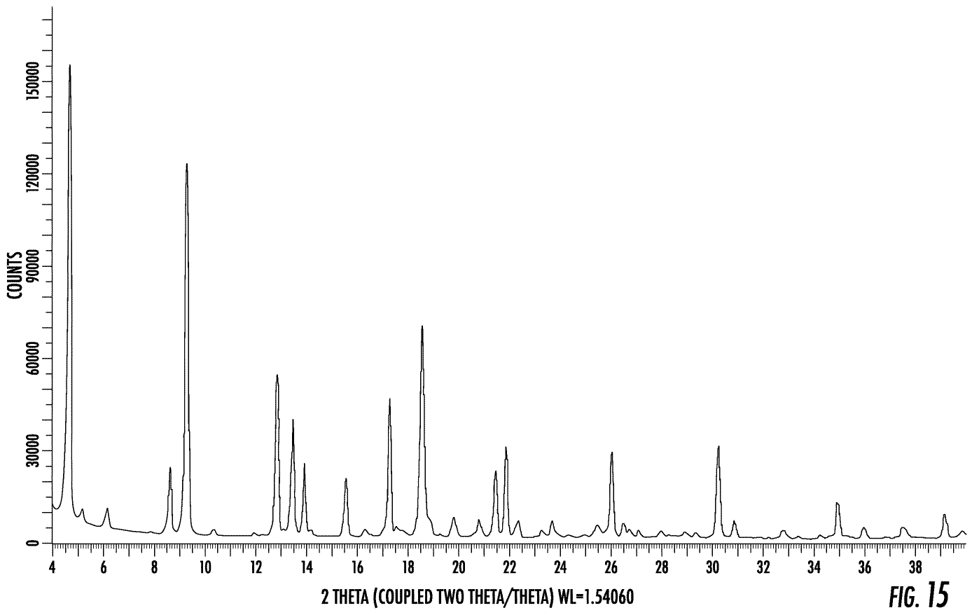

In a twenty-third embodiment, crystalline Form H is characterized by at least three, at least four, at least five, or at least six x-ray powder diffraction peaks at 2θ angles selected from 4.70°, 9.31°, 12.87°, 13.49°, 17.31°, 18.57°, and 21.88°. Alternatively, as part of a twenty-third embodiment, crystalline Form H is characterized by x-ray powder diffraction peaks at 2θ angles selected from 4.70°, 9.31°, 12.87°, 13.49°, 17.31°, 18.57°, and 21.88°. In another alternative, as part of a twenty-third embodiment, crystalline Form H is characterized by at least three, at least four, at least five, at least six, at least seven, at least eight, at least nine, at least ten, at least eleven, at least twelve, at least thirteen, or at least fourteen x-ray powder diffraction peaks at 2θ angles selected from those in Table 8. In yet another alternative, as part of a twenty-third embodiment, crystalline Form H is characterized by an XRPD substantially similar to FIG. 15.

In a twenty-fourth embodiment, crystalline Form H as described herein e.g., (in the twenty-second or twenty-third embodiment) is a solvate. Alternatively, as part of a twenty-fourth embodiment, crystalline Form H as described herein e.g., (in the twenty-second or twenty-third embodiment) is a DMSO solvate.

TABLE 8

| Position (°2θ) | Relative Intensity (%) |
|---|---|
| 4.70 | 100 |
| 5.21 | 2 |
| 6.19 | 5 |
| 8.66 | 16 |
| 9.31 | 90 |
| 12.87 | 40 |
| 13.12 | 2 |
| 13.49 | 29 |
| 13.95 | 18 |
| 14.19 | 2 |
| 15.58 | 15 |
| 16.34 | 2 |
| 17.31 | 35 |
| 17.58 | 3 |
| 18.57 | 52 |
| 18.84 | 4 |
| 19.83 | 5 |
| 20.81 | 5 |
| 21.47 | 17 |
| 21.88 | 24 |
| 22.37 | 4 |
| 22.37 | 4 |
| 23.29 | 2 |
| 23.69 | 5 |
| 25.50 | 3 |
| 26.04 | 23 |
| 26.52 | 4 |
| 26.76 | 2 |
| 27.11 | 2 |
| 28.01 | 2 |
| 28.92 | 2 |
| 30.24 | 26 |
| 30.88 | 5 |
| 32.79 | 2 |
| 34.94 | 11 |
| 35.98 | 3 |
| 37.51 | 3 |
| 39.15 | 7 |
| 22.37 | 4 |

In a twenty-fifth embodiment, provided is a crystalline form of a hydrochloric acid salt of a compound having the structural formula:

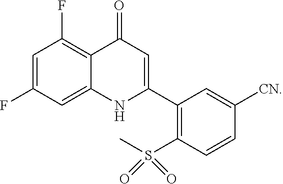 [SMILES: CS(=O)(=O)c1ccc(C#N)cc1-c1cc(=O)c2c(F)cc(F)cc2[nH]1]

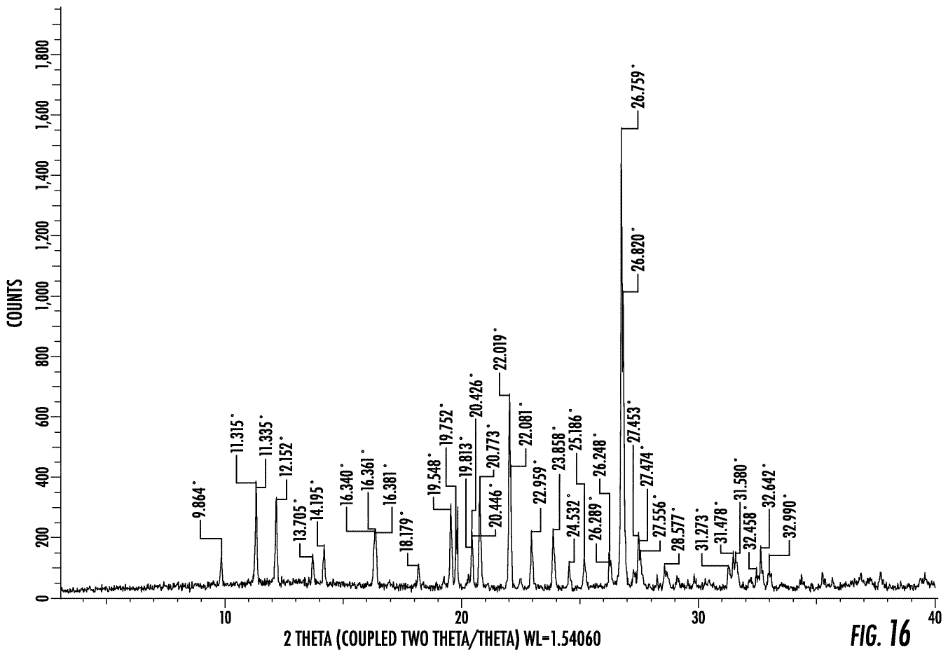

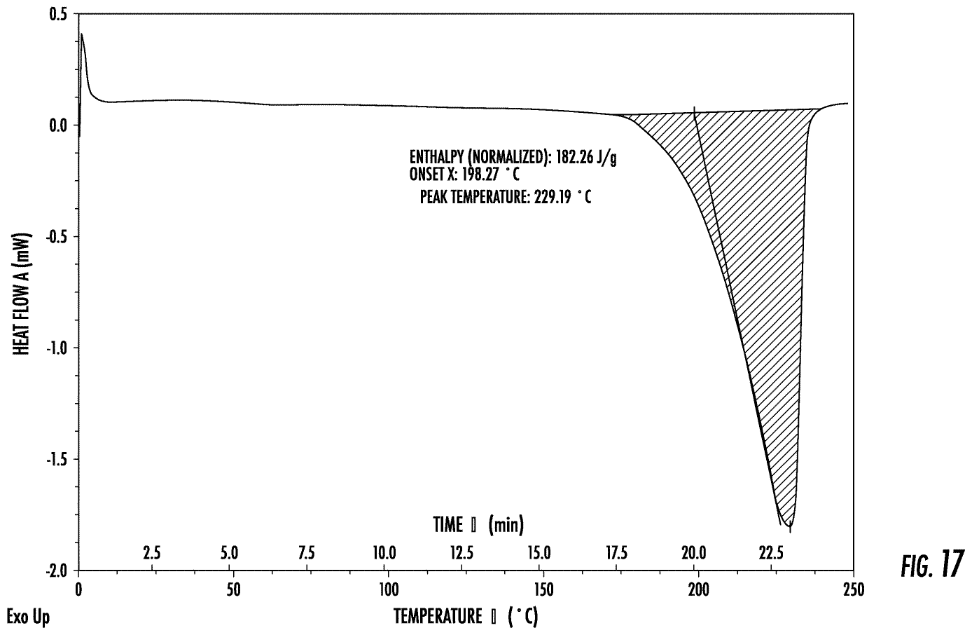

In a twenty-sixth embodiment, the crystalline form of the hydrochloric acid salt of Compound 1 is characterized by at least three, at least four, or at least five x-ray powder diffraction peaks at 2θ angles selected from 11.32°, 11.34°, 22.02°, 22.08°, 26.76°, and 26.82°. Alternatively, as part of a twenty-sixth embodiment, the crystalline form of the hydrochloric acid salt of Compound 1 is characterized by x-ray powder diffraction peaks at 2θ angles selected from 11.32°, 11.34°, 22.02°, 22.08°, 26.76°, and 26.82°. In another alternative, as part of a twenty-sixth embodiment, the crystalline form of the hydrochloric acid salt of Compound 1 is characterized by x-ray powder diffraction peaks at 2θ angles selected from 11.32°, 11.34°, 22.02°, 22.08°, 26.76°, and 26.82°, and at least one, at least two, at least three, at least four, at least five, at least six, at least seven, or at least eight x-ray powder diffraction peaks at 2θ angles selected 12.15°, 16.34°, 16.36°, 16.38°, 19.54°, 19.75°, 20.46°, 20.45°, and 20.77°. In yet another alternative, as part of a twenty-sixth embodiment, the crystalline form of the hydrochloric acid salt of Compound 1 is characterized by x-ray powder diffraction peaks at 2θ angles selected from 11.32°, 11.34°, 12.15°, 16.34°, 16.36°, 16.38°, 19.54°, 19.75°, 20.46°, 20.45°, 20.77°, 22.02°, 22.08°, 26.76°, and 26.82°. In yet another alternative, as part of a twenty-sixth embodiment, the crystalline form of the hydrochloric acid salt of Compound 1 is characterized by at least three, at least four, at least five, at least six, at least seven, at least eight, at least nine, at least ten, at least eleven, at least twelve, at least thirteen, or at least fourteen x-ray powder diffraction peaks at 2θ angles selected from those in Table 9. In yet another alternative, as part of a twenty-sixth embodiment, the crystalline form of the hydrochloric acid salt of Compound 1 is characterized by an XRPD substantially similar to FIG. 16. In yet another alternative, as part of a twenty-sixth embodiment, the crystalline form of the hydrochloric acid salt of Compound 1 is characterized by a DSC substantially similar to FIG. 17.

TABLE 9

| Position (°2θ) | Relative Intensity (%) |
|---|---|
| 9.864 | 8.20 |
| 11.315 | 22.50 |
| 11.335 | 21.00 |
| 12.152 | 17.90 |
| 13.705 | 6.50 |
| 14.195 | 8.60 |
| 16.34 | 12.20 |
| 16.361 | 12.60 |
| 16.381 | 11.90 |
| 18.179 | 5.10 |
| 19.548 | 18.30 |
| 19.752 | 13.80 |
| 19.813 | 9.20 |
| 20.426 | 13.10 |
| 20.446 | 12.10 |
| 20.773 | 19.20 |
| 22.019 | 42.30 |
| 22.081 | 26.90 |
| 22.959 | 12.50 |
| 23.858 | 13.00 |
| 24.532 | 5.90 |
| 25.186 | 6.80 |
| 26.248 | 7.80 |
| 26.289 | 5.60 |
| 26.759 | 100.00 |
| 26.82 | 64.40 |
| 27.453 | 10.70 |
| 27.474 | 11.80 |
| 27.556 | 8.00 |
| 28.577 | 5.40 |
| 31.273 | 4.80 |
| 31.478 | 7.90 |
| 31.58 | 7.70 |
| 32.458 | 4.40 |
| 32.642 | 9.00 |
| 32.99 | 7.30 |

In an twenty-seventh embodiment, provided is a crystalline form of a methanesulfonic acid salt of a compound having the structural formula:

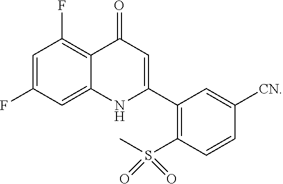 [SMILES: CS(=O)(=O)c1ccc(C#N)cc1-c1cc(=O)c2c(F)cc(F)cc2[nH]1]

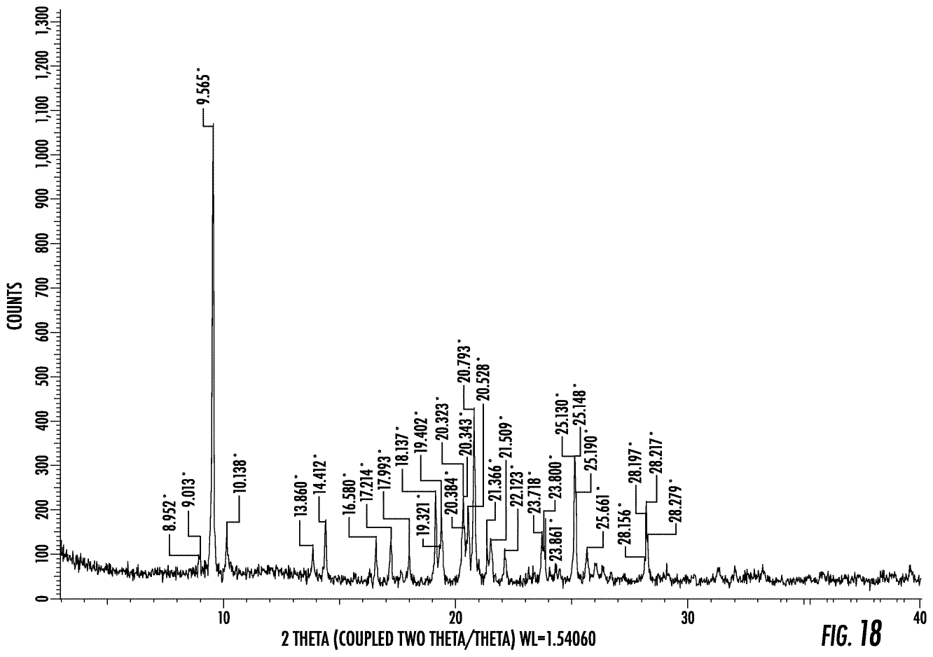

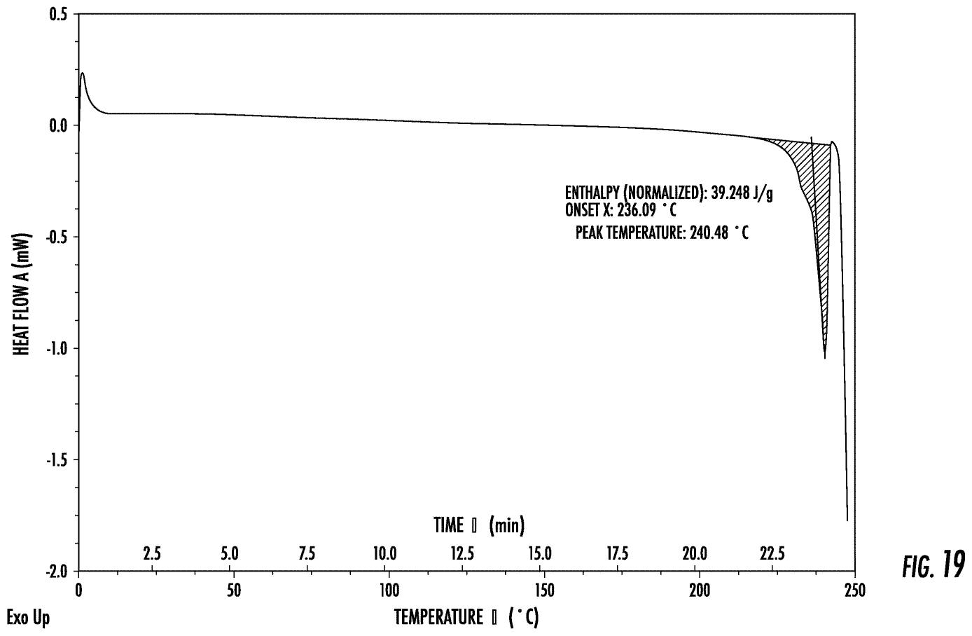

In a twenty-eighth embodiment, the crystalline form of the methanesulfonic acid salt of Compound 1 is characterized by at least three or at least four x-ray powder diffraction peaks at 2θ angles selected from 9.57°, 19.14°, 20.79°, 25.13°, and 25.15°. Alternatively, as part of a twenty-eighth embodiment, the crystalline form of the methanesulfonic acid salt of Compound 1 is characterized by x-ray powder diffraction peaks at 2θ angles selected from 9.57°, 19.14°, 20.79°, 25.13°, and 25.15°. In another alternative, as part of a twenty-eighth embodiment, the crystalline form of the methanesulfonic acid salt of Compound 1 is characterized by x-ray powder diffraction peaks at 2θ angles selected from 9.57°, 19.14°, 20.79°, 25.13°, and 25.15°, and at least one, at least two, at least three, at least four, at least five, at least six, at least seven, at least eight, at least nine, at least ten, at least eleven, or at least twelve additional x-ray powder diffraction peaks at 2θ angles selected from 14.41°, 16.58°, 17.21°, 19.40°, 20.32°, 20.34°, 20.38°, 20.53°, 23.72°, 25.19°, 28.20°, 28.22°, and 28.28°. In yet another alternative, as part of a twenty-eighth embodiment, the crystalline form of the methanesulfonic acid salt of Compound 1 is characterized by at least three, at least four, at least five, at least six, at least seven, at least eight, at least nine, at least ten, at least eleven, at least twelve, at least thirteen, or at least fourteen x-ray powder diffraction peaks at 2θ angles selected from those in Table 10. In yet another alternative, as part of a twenty-eighth embodiment, the crystalline form of the methanesulfonic acid salt of Compound 1 is characterized by an XRPD substantially similar to FIG. 18. In yet another alternative, as part of a twentieth embodiment, the crystalline form of the methanesulfonic acid salt of Compound 1 is characterized by a DSC substantially similar to FIG. 19.

TABLE 10

| Position (°2θ) | Relative Intensity (%) |
|---|---|
| 8.952 | 4.50 |
| 9.013 | 4.80 |
| 9.565 | 100.00 |
| 10.138 | 8.90 |
| 13.86 | 7.40 |
| 14.412 | 13.20 |
| 16.58 | 10.00 |
| 17.214 | 10.30 |
| 17.993 | 6.40 |
| 19.137 | 20.20 |
| 19.321 | 8.30 |
| 19.402 | 14.10 |
| 20.323 | 18.70 |
| 20.343 | 17.00 |
| 20.384 | 12.10 |
| 20.528 | 16.40 |
| 20.793 | 38.10 |
| 21.366 | 7.80 |
| 21.509 | 9.20 |
| 22.123 | 7.10 |
| 23.718 | 10.90 |
| 23.8 | 8.60 |
| 23.861 | 5.20 |
| 25.13 | 27.50 |
| 25.148 | 27.30 |
| 25.19 | 19.40 |
| 25.661 | 7.50 |
| 28.156 | 5.30 |
| 28.197 | 14.80 |
| 28.217 | 17.50 |
| 28.279 | 10.40 |

In a twenty-first embodiment, provided are pharmaceutically acceptable compositions comprising a described crystalline form (e.g., as in any one of the first to twentieth embodiments) together with a pharmaceutically acceptable carrier.

### Uses, Formulation and Administration

The crystalline forms and compositions described herein are generally useful for modulating the activity of PPARG. In some aspects, the crystalline forms and pharmaceutical acceptable salts disclosed herein are inverse agonists of PPARG. In one aspect, “inverse-agonists” refer to agents that bind to the same receptor binding site as an agonist (e.g., the binding site of a nuclear receptor such as PPARG) and not only antagonizes the effects of an agonist but, moreover, exerts the opposite effect by suppressing spontaneous receptor signaling (when present).

In some aspects, the crystalline forms and pharmaceutical compositions described herein are useful in treating a disorder associated with PPARG function. Thus, provided herein are methods of treating a disorder associated with PPARG function, comprising administering to a subject in need thereof, a therapeutically effective amount of a crystalline form described herein or a pharmaceutical composition comprising a disclosed crystalline form.

Also provided is the use of a crystalline form described herein, or a pharmaceutical composition comprising a disclosed crystalline form, for the manufacture of a medicament for treating a disorder associated with PPARG function. Also provided is a crystalline form described herein or a pharmaceutical composition comprising a disclosed crystalline form for use in treating a disorder associated with PPARG.

In one aspect, the disorder associated with PPARG is cancer. In some aspects, the cancer is associated with an up-regulated peroxisome proliferator-activated receptor (PPAR) signaling pathway. In some aspects, the up-regulated PPAR signaling pathway is associated with increased expression of one or more genes selected from Uroplakin 1A (UPK1A), Uroplakin IB (UPK1B), Uroplakin (UPK2), Keratin 20 (KRT20), GATA Binding Protein 3 (GAT A3), Nuclear Receptor Corepressor 1 (NCOR1), Nuclear Receptor Corepressor 2 (NCOR2), Fatty Acid Binding Protein 4 (FABP4), Forkhead Box A1 (FOXA1), CD36 Molecule (CD36), Acyl-CoA Oxidase 1 (ACOX1), 3-Hydroxy-3-Methylglutaryl-CoA Synthase 2 (HMGCS2), Acyl-CoA Synthetase Long-Chain Family Member 5 (ACSL5), Arachidonate 5-Lipoxygenase (ALOX5), Acyl-CoA Synthetase Long-Chain Family Member 1 (ACSL1), and Angiopoietin Like 4 (ANGPTL4).

In some aspects, the cancer treated is selected from breast cancer, pancreatic cancer, ovarian cancer, prostate cancer, renal cancer, bladder cancer, testicular cancer, urothelial cancer (e.g., non-muscle-invasive urothelial cancer, muscle-invasive urothelial cancer, metastatic urothelial cancer), skin cancer, melanoma, colon cancer, kidney cancer, brain cancer and a hematopoietic cancer (e.g., lymphoma, multiple myeloma and leukemia). In one aspect, the cancer treated is urothelial cancer such as non-muscle-invasive urothelial cancer, muscle-invasive urothelial cancer, and metastatic urothelial cancer.

Other uses besides cancer are contemplated and include e.g., metabolic diseases (e.g., osteoporosis, rachitis, arthrosis, obesity, type I and type II diabetes mellitus), lipid metabolism disorder, pancreatitis, glucose metabolism disorder, diabetic neuropathy, diabetic complications, hyperuricemia, osteoporosis, rachitis, arthrosis inflammatory diseases (e.g., inflammatory skin diseases such as psoriasis, atopic dermatitis, eczema, acne vulgaris, other dermatitides and pruritus), pulmonary disorders (e.g., asthma and chronic obstructive pulmonary disease), autoimmune disease, neurodegenerative disease (e.g., multiple sclerosis, Alzheimer's disease, and Parkinson's disease), cardiovascular diseases (e.g., selected from atherosclerosis, venous and arterial occlusive diseases), restenosis after invasive procedures, cardiomyopathy, myocardial fibrosis, congestive heart failure, angiogenesis and neovascularization in neoplastic diseases and renal diseases.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

EXEMPLIFICATION

The representative examples that follow are intended to help illustrate the present disclosure, and are not intended to, nor should they be construed to, limit the scope of the invention.

Crystalline Form A of 3-(5,7-difluoro-4-oxo-1,4-dihydroquinolin-2-yl)-4-(methylsulfonyl)benzonitrile was prepared according to the procedures described for Example 1 in PCT/CN2022/129034.

XRPD samples were analyzed using a Bruker D8 ADVANCE X-ray diffractometer using copper radiation (Cu Kα, λ=1.54060 Å). The generator was operated at a voltage of 40 kV and an amperage of 40 mA. Data were collected with a scanning range of 3 to 40° with a step size of 0.02°, scanning speed of 10°/minute and a sample rotation speed of 15 rpm.

DSC was performed using a TA Instruments Discovery 2500. The sample (0.5-1.5 mg) was weighed directly in a Tzero pan and Tzero hermetic lid with a pin hole of 0.7 mm from diameter and analyzed according to the parameters below:

| Method | Ramp | |
|---|---|---|
| Sample size | 0.5-1.5 | mg |
| Heating rate | 10.0 | °C/min |
| Temperature range | 0 to 350 | °C |
| Method gas | N₂ at 50.00 | mL/min |

### General Procedure for Obtaining Crystalline Forms

About 2 g of crystalline Form A was ground manually with a mortar and pestle for approximately 4 hours. Approximately 40 mg of the resulting material was stirred in 0.2-0.6 mL of solvent at 25° C. for 2 weeks with a stirring bar on a magnetic stirring plate at a rate of 300-400 rpm. Obtained suspensions were filtered through a 0.45 μm nylon membrane filter by centrifugation at 14,000 rpm. Solid parts were then analyzed via XRPD, DSC, TGA, etc.

Crystalline Form B was obtained using MeOH as the solvent in the above general procedure.

Crystalline Form C was obtained using water (at 50° C. for approximately 1 week) as the solvent in the above general procedure.

Crystalline Form D was obtained using MTBE as the solvent in the above general procedure.

Crystalline Form E was obtained by heating Pattern C to 300° C. by TGA (10° C./min), and cooled to about 25° C.

Crystalline Form F was obtained by adding 2-MeTHF (1.0 mL) to a mixture of crystalline Form A and crystalline Form B (50 mg total), which was then stirred with a stirbar at 20-30° C. (also at 45-55° C.) for approximately 16 hours. Crystalline Form F was filtered and isolated.

Crystalline Form H was prepared by taking approximately 1.4 g of Form C was weighed into each 20 mL vial. The solid was dissolved in 10 mL DMSO and a stir bar was added. Additional Form C were added to the vial to increase the concentration of the solution. The solution was then syringe filtered and the vial was then stirred at 50° C. and left uncapped to reduce the volume of DMSO. An off-white slurry was observed and XRPD confirmed the formation of Pattern H. Pattern H could only be isolated as a wet cake.

Pattern G′ was obtained after treating Pattern H with an EtOAc wash (3 mL×2 aliquots) and drying at room temperature under active vacuum (−29 inHg) overnight. Pattern H converts to G′ which appeared to be a possible intermediate between Pattern H and Pattern G. Upon further drying (50° C., vacuum oven) Pattern G′ converted to Pattern G.

### General Procedure for Obtaining Crystalline Salt Forms

About 50 mg of crystalline Form A, and 1.0 equivalent of base, coformer or acid were added into a screening solvent in a 2 mL glass vial and stir bar. Obtained mixtures were stirred at 50° C. with a stirring bar on a magnetic stirring plate at a rate of 400 rpm for about 1 day and then at 25° C. for about 3 days.

The crystalline hydrochloride salt of 3-(5,7-difluoro-4-oxo-1,4-dihydroquinolin-2-yl)-4-(methylsulfonyl)benzonitrile was obtained from DMSO/acetone (1/2 v/v, 0.3 mL). The crystalline methanesulfonic acid salt of 3-(5,7-difluoro-4-oxo-1,4-dihydroquinolin-2-yl)-4-(methylsulfonyl)benzonitrile was obtained from THF (0.4 mL) or DMSO/acetone (1/2 v/v, 0.45 mL).

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. Crystalline Form B, C, D, E, F, G, G′, or H of a compound having the structural formula:

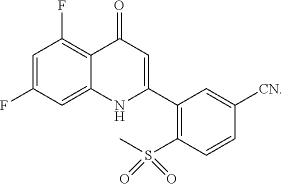 [SMILES: CS(=O)(=O)c1ccc(C#N)cc1-c1cc(=O)c2c(F)cc(F)cc2[nH]1]

2. The crystalline Form B of claim 1, wherein the crystalline form is characterized by:
(i) at least three x-ray powder diffraction peaks at 2θ angles selected from 11.74°, 13.27°, 19.51°, 21.78°, 26.30°, and 27.26°;
(ii) at least four x-ray powder diffraction peaks at 2θ angles selected from 11.74°, 13.27°, 19.51°, 21.78°, 26.30°, and 27.26°;
(iii) at least five x-ray powder diffraction peaks at 2θ angles selected from 11.74°, 13.27°, 19.51°, 21.78°, 26.30°, and 27.26°;
(iv) x-ray powder diffraction peaks at 2θ angles selected from 11.74°, 13.27°, 19.51°, 21.78°, 26.30°, and 27.26°;
(v) x-ray powder diffraction peaks at 2θ angles selected from 11.74°, 13.27°, 19.51°, 21.78°, 26.30°, and 27.26°, and at least one, at least two, at least three, at least four, at least five, at least six, at least seven, at least eight, or at least nine additional x-ray powder diffraction peaks at 2θ angles selected from 7.78°, 16.28°, 17.68°, 18.25°, 18.91°, 20.58°, 23.35°, 25.45°, 28.60°, and 29.93°; or
(vi) x-ray powder diffraction peaks at 2θ angles selected from 7.78°, 11.74°, 13.27°, 16.28°, 17.68°, 18.25°, 18.91°, 19.51°, 20.58°, 21.78°, 23.35°, 25.45°, 26.30°, 27.26°, 28.60°, and 29.93°.

3-10. (canceled)

11. The crystalline Form C of claim 1, wherein the crystalline form is characterized by:
(i) at least three x-ray powder diffraction peaks at 2θ angles selected from 14.31°, 16.21°, 16.32°, 17.15°, 21.82°, and 26.90°;
(ii) at least four x-ray powder diffraction peaks at 2θ angles selected from 14.31°, 16.21°, 16.32°, 17.15°, 21.82°, and 26.90°;
(iii) at least five x-ray powder diffraction peaks at 2θ angles selected from 14.31°, 16.21°, 16.32°, 17.15°, 21.82°, and 26.90°;
(iv) x-ray powder diffraction peaks at 2θ angles selected from 14.31°, 16.21°, 16.32°, 17.15°, 21.82°, and 26.90°;
(v) x-ray powder diffraction peaks at 2θ angles selected from 14.31°, 16.21°, 16.32°, 17.15°, 21.82°, and 26.90° and at least one, at least two, at least three, at least four, at least five, at least six, at and least seven, additional x-ray powder diffraction peaks at 2θ angles selected from 18.34°, 19.85°, 20.46°, 24.23°, 25.86°, 27.38°, 27.89°, and 29.34°; or
(vi) x-ray powder diffraction peaks at 2θ angles selected from 14.31°, 16.21°, 16.32°. 17.15°, 18.34°, 19.85°, 20.46°, 21.82°, 24.23°, 25.86°, 26.90°, 27.38°, 27.89°, and 29.34°.

12-18. (canceled)

19. The crystalline Form D of claim 1, wherein the crystalline form is characterized by:
(i) at least three x-ray powder diffraction peaks at 2θ angles selected from 8.44°, 16.58°, 18.73°, 19.10°, 23.31°, 25.36°, and 26.45°;
(ii) at least four x-ray powder diffraction peaks at 2θ angles selected from 8.44°, 16.58°, 18.73°, 19.10°, 23.31°, 25.36°, and 26.45°;
(iii) at least five x-ray powder diffraction peaks at 2θ angles selected from 8.44°, 16.58°, 18.73°, 19.10°, 23.31°, 25.36°, and 26.45°;
(iv) at least six x-ray powder diffraction peaks at 2θ angles selected from 8.44°, 16.58°, 18.73°, 19.10°, 23.31°, 25.36°, and 26.45°; or
(v) x-ray powder diffraction peaks at 2θ angles selected from 8.44°, 16.58°, 18.73°, 19.10°, 23.31°, 25.36°, and 26.45°.

20-26. (canceled)

27. The crystalline Form E of claim 1, wherein the crystalline form is characterized by:
(i) at least three x-ray powder diffraction peaks at 2θ angles selected from 22.68°, 22.73°, 24.27°, 26.22°, and 26.29°;
(ii) at least four x-ray powder diffraction peaks at 2θ angles selected from 22.68°, 22.73°. 24.27°, 26.22°, and 26.29°;
(iii) x-ray powder diffraction peaks at 2θ angles selected from 22.68°, 22.73°, 24.27°, 26.22°, and 26.29°;
(iv) x-ray powder diffraction peaks at 2θ angles selected from 22.68°, 22.73°, 24.27°, 26.22°, and 26.29°, and at least one, at least two, at least, or at least four additional x-ray powder diffraction peaks at 2θ angles selected from 5.66°, 11.30°, 12.09°, 24.33°, and 29.19°; or
(v) x-ray powder diffraction peaks at 2θ angles selected from 5.66°, 11.30°, 12.09°, 22.68°, 22.73°, 24.27°, 24.33°, 26.22°, 26.29°, and 29.19°.

28-33. (canceled)

34. The crystalline Form F of claim 1, wherein the crystalline form is characterized by:
(i) at least three x-ray powder diffraction peaks at 2θ angles selected from 8.51°, 10.84°, 16.49°, 17.67°, 18.85°, 20.92°, and 25.60°;
(ii) at least four x-ray powder diffraction peaks at 2θ angles selected from 8.51°, 10.84°, 16.49°, 17.67°, 18.85°, 20.92°, and 25.60°;
(iii) at least five x-ray powder diffraction peaks at 2θ angles selected from 8.51°, 10.84°, 16.49°, 17.67°, 18.85°, 20.92°, and 25.60°;
(iv) at six three x-ray powder diffraction peaks at 2θ angles selected from 8.51°, 10.84°, 16.49°, 17.67°, 18.85°, 20.92°, and 25.60°; or
(v) x-ray powder diffraction peaks at 2θ angles selected from 8.51°, 10.84°, 16.49°, 17.67°, 18.85°, 20.92°, and 25.60°.

35-41. (canceled)

42. The crystalline Form G of claim 1, wherein the crystalline form is characterized by:
(i) at least three x-ray powder diffraction peaks at 2θ angles selected from 4.64°, 9.27°, 16.98°, 18.30°, 18.42°, 18.97°, and 25.29°;
(ii) at least four x-ray powder diffraction peaks at 2θ angles selected from 4.64°, 9.27°, 16.98°, 18.30°, 18.42°, 18.97°, and 25.29°;
(iii) at least five x-ray powder diffraction peaks at 2θ angles selected from 4.64°, 9.27°, 16.98°, 18.30°, 18.42°, 18.97°, and 25.29°;
(iv) at least six x-ray powder diffraction peaks at 2θ angles selected from 4.64°, 9.27°, 16.98°, 18.30°, 18.42°, 18.97°, and 25.29°; or
(v) x-ray powder diffraction peaks at 2θ angles selected from 4.64°, 9.27°, 16.98°, 18.30°, 18.42°, 18.97°, and 25.29°.

43-49. (canceled)

50. The crystalline Form G′ of claim 1, wherein the crystalline form is characterized by:
(i) at least three x-ray powder diffraction peaks at 2θ angles selected from 4.65°, 9.26°, 15.55°, 18.31°, 18.87°, 25.27°, and 26.41°;
(ii) at least four x-ray powder diffraction peaks at 2θ angles selected from 4.65°, 9.26°, 15.55°, 18.31°, 18.87°, 25.27°, and 26.41°;
(iii) at least five x-ray powder diffraction peaks at 2θ angles selected from 4.65°, 9.26°, 15.55°, 18.31°, 18.87°, 25.27°, and 26.41°;
(iv) at least six x-ray powder diffraction peaks at 2θ angles selected from 4.65°, 9.26°, 15.55°, 18.31°, 18.87°, 25.27°, and 26.41°; or
(v) x-ray powder diffraction peaks at 2θ angles selected from 4.65°, 9.26°, 15.55°, 18.31°, 18.87°, 25.27°, and 26.41°.

51-57. (canceled)

58. The crystalline Form H of claim 1, wherein the crystalline form is characterized by:
(i) at least three x-ray powder diffraction peaks at 2θ angles selected from 4.70°, 9.31°, 12.87°, 13.49°, 17.31°, 18.57°, and 21.88°;
(ii) at least four x-ray powder diffraction peaks at 2θ angles selected from 4.70°, 9.31°, 12.87°, 13.49°, 17.31°, 18.57°, and 21.88°;
(iii) at least five x-ray powder diffraction peaks at 2θ angles selected from 4.70°, 9.31°, 12.87°, 13.49°, 17.31°, 18.57°, and 21.88°;
(iv) at least six x-ray powder diffraction peaks at 2θ angles selected from 4.70°, 9.31°, 12.87°, 13.49°, 17.31°, 18.57°, and 21.88°; or
(v) x-ray powder diffraction peaks at 2θ angles selected from 4.70°, 9.31°, 12.87°, 13.49°, 17.31°, 18.57°, and 21.88°.

59-65. (canceled)

66. A crystalline form of a hydrochloric acid salt of a compound having the structural formula:

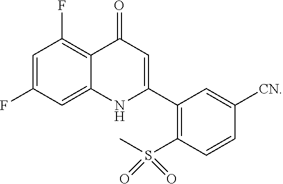 [SMILES: CS(=O)(=O)c1ccc(C#N)cc1-c1cc(=O)c2c(F)cc(F)cc2[nH]1]

67. The crystalline form of the hydrochloric acid salt of the compound of claim 66, wherein the crystalline form is characterized by:
(i) at least three x-ray powder diffraction peaks at 2θ angles selected from 11.32°, 11.34°, 22.02°, 22.08°, 26.76°, and 26.82°;
(ii) at least four x-ray powder diffraction peaks at 2θ angles selected from 11.32°, 11.34°, 22.02°, 22.08°, 26.76°, and 26.82°;
(iii) at least five x-ray powder diffraction peaks at 2θ angles selected from 11.32°, 11.34°, 22.02°, 22.08°, 26.76°, and 26.82°;
(iv) x-ray powder diffraction peaks at 2θ angles selected from 11.32°, 11.34°, 22.02°, 22.08°, 26.76°, and 26.82°;
(v) x-ray powder diffraction peaks at 2θ angles selected from 11.32°, 11.34°, 22.02°, 22.08°, 26.76°, and 26.82°, and at least one, at least two, at least three, at least four, at least five, at least six, at least seven, or at least eight x-ray powder diffraction peaks at 2θ angles selected 12.15°, 16.34°, 16.36°, 16.38°, 19.54°, 19.75°, 20.46°, 20.45°, and 20.77°; or
(vi) x-ray powder diffraction peaks at 2θ angles selected from 11.32°, 11.34°, 12.15°, 16.34°, 16.36°, 16.38°, 19.54°, 19.75°, 20.46°, 20.45°, 20.77°, 22.02°, 22.08°, 26.76°, and 26.82°.

68-73. (canceled)

74. A crystalline form of a methanesulfonic acid salt of a compound having the structural formula:

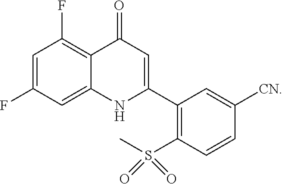 [SMILES: CS(=O)(=O)c1ccc(C#N)cc1-c1cc(=O)c2c(F)cc(F)cc2[nH]1]

75. The crystalline form of the methanesulfonic acid salt of the claim 74, wherein the crystalline form is characterized by:
(i) at least three x-ray powder diffraction peaks at 2θ angles selected from 9.57°, 19.14°, 20.79°, 25.13°, and 25.15°;
(ii) at least four x-ray powder diffraction peaks at 2θ angles selected from 9.57°, 19.14°, 20.79°, 25.13°, and 25.15°;
(iii) x-ray powder diffraction peaks at 2θ angles selected from 9.57°, 19.14°, 20.79°, 25.13°, and 25.15°;
(iv) x-ray powder diffraction peaks at 2θ angles selected from 9.57°, 19.14°, 20.79°, 25.13°, and 25.15°, and at least one, at least two, at least three, at least four, at least five, at least six, at least seven, at least eight, at least nine, at least ten, at least eleven, or at least twelve additional x-ray powder diffraction peaks at 2θ angles selected from 14.41°, 16.58°, 17.21°, 19.40°, 20.32°, 20.34°, 20.38°, 20.53°, 23.72°, 25.19°, 28.20°, 28.22°, and 28.28°; or
(v) x-ray powder diffraction peaks at 2θ angles selected from 9.57°, 14.41°, 16.58°, 17.21°, 19.14°, 19.40°, 20.32°, 20.34°, 20.38°, 20.53°, 20.79°, 23.72°, 25.13°, 25.19°, 25.15°, 28.20°, 28.22°, and 28.28°,

76-80. (canceled)

81. A pharmaceutical composition comprising the crystalline Form B, C, D, E, F, G, G′, or H of claim 1 and a pharmaceutically acceptable carrier or diluent.

82. A method of treating a cancer responsive to the suppression of PPARG in a subject, comprising administering to the subject a therapeutically effective amount of the crystalline Form B, C, D, E, F, G, G′, or H of claim 1.

83. The method of claim 82, wherein the cancer is selected from breast cancer, pancreatic cancer, ovarian cancer, prostate cancer, renal cancer, bladder cancer, testicular cancer, urothelial cancer, skin cancer, melanoma, colon cancer, kidney cancer, brain cancer and a hematopoietic cancer.

84. The method of claim 82, wherein the cancer is bladder cancer.
